(19) United States
(12) Patent Application Publication
(10) Pub. No.: US 2014/0350623 A1
(43) Pub. Date: Nov. 27, 2014

(54) SYSTEM AND METHOD FOR CONTROLLING ELECTRICAL STIMULATION BASED ON LOWEST OPERABLE VOLTAGE MULTIPLIER FOR USE WITH IMPLANTABLE MEDICAL DEVICE

(71) Applicant: PACESETTER, INC., Sylmar, CA (US)

(72) Inventors: Avi Fischer, Los Angeles, CA (US); Bryan Tilton, Highlands Ranch, CO (US); Gene A. Bornzin, Simi Valley, CA (US)

(73) Assignee: PACESETTER, INC., Sylmar, CA (US)

(21) Appl. No.: 13/901,937

(22) Filed: May 24, 2013

(51) Int. Cl. A61N 1/365; A61N 1/37; A61N 1/39

(52) U.S. Cl. CPC A61N 1/365; A61N 1/3956; A61N 1/3712
USPC 607/11; 607/6

(57) ABSTRACT

Techniques are provided for use with implantable devices equipped with programmable voltage multipliers (including voltage dividers.) Candidate pulse widths are determined for selected voltage multipliers and stimulation vectors. Each candidate pulse width corresponds to a lowest pulse energy sufficient to achieve capture within the tissues of the patient (subject to a safety margin) using the selected vector and using the corresponding voltage multiplier. As such, a candidate pulse width represents a preferred or optimal pulse width, at least insofar as energy consumption is concerned. However, depending upon the capabilities of the device, the candidate pulse width might not be achievable. Accordingly, for each programmable vector, the system determines a lowest “operable” voltage multiplier sufficient to generate a pulse at a candidate pulse width subject to the capabilities of the device. The system then determines the corresponding current drain, and the vector achieving the lowest current drain at the lowest operable voltage multiplier is selected for the delivery of stimulation.

### FIELD OF THE INVENTION

The invention relates to implantable cardiac rhythm management devices (CRMDs) such as pacemakers, cardiac resynchronization therapy (CRT) devices and implantable cardioverter-defibrillators (ICDs) and, in particular, to techniques for minimizing current drain within such devices.

### BACKGROUND OF THE INVENTION

State-of-the-art CRT devices and lead multi-pole systems—such as the Quadra™ CRT-D and the Quartet™ left ventricular (LV) lead of St. Jude Medical—have enabled clinicians to non-invasively reprogram LV pacing vectors to optimize pacing location, mitigate phrenic (i.e. diaphragmatic) nerve stimulation, and choose pacing parameters for reduced power consumption. See, for example, systems and methods described in: U.S. Pat. No. 8,209,010, U.S. Published Application 2011/0196442, each to Ryu et al. See, also, U.S. Pat. No. 5,697,956 to Bornzin, which describes an implantable pacemaker that maintains a prescribed relationship between stimulation pulse amplitude and pulse width so as to provide an adequate safety factor above a stimulation threshold while minimizing the current drain on the pacemaker battery. A series of stimulation pulse energies, each realized with a prescribed pulse amplitude and pulse width pair, are determined that may be used by the pacemaker as operating points. The operating points are numbered in order of increasing energy and adjustments to the pacing energy are made by selecting one of these operating points.

Although techniques described in these documents are helpful, further room for improvement remains in providing systems that quickly and efficiently identify particular combinations of pacing parameters—including pacing vector combinations, pulse widths, pulse voltages, etc.—that provide for reduced battery consumption, particularly within systems equipped with numerous programmable stimulation vectors or vector combinations.

It is to these general ends that the invention is directed.

### SUMMARY OF THE INVENTION

In an exemplary embodiment, a method is provided for use with an implantable medical device such as a CRMD equipped to deliver stimulation along one or more programmable stimulation vectors and equipped with one or more voltage multipliers (including voltage dividers.) Briefly, for a selected programmable stimulation vector, the system determines candidate pulse widths (herein PWopt values) for selected voltage multipliers. Each candidate pulse width corresponds to a lowest pulse energy sufficient to achieve capture within the tissues of the patient (subject to a programmable safety factor) using the selected vector and using the voltage supplied by a corresponding voltage multiplier. As such, a candidate pulse width represents a preferred or optimal pulse width, at least insofar as energy consumption is concerned. However, depending upon the capabilities of the device, the candidate pulse width corresponding to a particular voltage multiplier for a particular stimulation vector might not be achievable because it is, e.g., too short. Accordingly, the system determines a lowest “operable” voltage multiplier for the selected vector, wherein the lowest operable voltage multiplier represents the lowest multiplier sufficient to provide enough voltage to generate a pulse at the candidate pulse width, at least within a range of programmable pulse widths supported by the device.

The exemplary system then controls the generation of stimulation pulses using the candidate pulse width corresponding to the lowest operable voltage multiplier for delivery along the selected vector, thereby reducing or minimizing power drain. Assuming the implantable device is equipped with multiple programmable stimulation vectors, the procedure is applied to determine the lowest operable voltage multiplier (and corresponding PWopt value) for each of the programmable vectors (or vector combinations), then the pulse charge corresponding to that pulse width and multiplier is determined (referred to herein as Qbat_opt) for each vector (or vector combination). The vector (or vector combination) having the lowest Qbat_opt value is selected for delivery of stimulation pulses to further minimize battery drain and improve device longevity. The procedure may be performed by a device programmer or other external system used to program the implanted device under clinical supervision or, if so equipped, the procedure may be performed by the implanted device itself.

In an illustrative implementation, the PWopt value for a selected vector is determined by first determining rheobase (Rho) and chronaxie (Chron) values for the vector by determining capture thresholds (Vth1, Vth2) at two different pulse widths (PW1, PW2) and then using:

$$Rho = \frac{{V}_{th 2} - \frac{{PW}_{1}}{{PW}_{2}} \cdot {V}_{th 1}}{1 - \frac{{PW}_{1}}{{PW}_{2}}}$$

$$and$$

$$Chron = \frac{( {V}_{th 1} - Rho ) \cdot {PW}_{1}}{Rho} .$$

Then, for a selected multiplier value (Mult), which may be ¼, ½, 1, 2, 3, etc., (depending upon the capabilities of the device), the system determines PWopt for the selected vector based on the rheobase and chronaxie values and a voltage (Vbat) of the voltage source of the device using:

$${PW}_{opt} = \frac{Chron}{\frac{{V}_{stim}}{Margin \cdot Rho} - 1} {V}_{stim}$$

where Vstim is the currently selected multiplier value (Mult) times Vbat and Margin is an optional safety factor. A typical Vbat value is 3.2 volts. A typical Margin is 1.25 to 2.5, depending on whether the implanted device is in an acute post-implant phase and whether automatic capture verification is implemented.

In the illustrative implementation, the system then determines the lowest operable voltage multiplier by identifying the lowest Mult value that provides a PWopt value within a range of programmable pulse widths, such as within a range from 0.05 milliseconds (ms) to 1.0 ms. This lowest Mult value is then designated, denoted or otherwise recorded as the lowest “operable” voltage multiplier for the selected vector. For example, within a implantable device equipped with a voltage halver, a voltage quarterer, a voltage doubler and a voltage tripler, the programmable voltage multiplier values (Mutt) of the device are ¼, ½, 1, 2 and 3. Within such an embodiment, if the voltage quarterer is found to provide a candidate pulse width (PWopt) within the programmable range of pulse widths, then the lowest operable voltage multiplier value for the vector is ¼. If the voltage quarterer is not capable of providing a PWopt value within the programmable range of pulse widths but the voltage halver is capable, then the lowest operable voltage multiplier value for the selected vector is ½.

Preferably, the assessment procedure for a selected vector begins with the lowest programmable voltage multiplier (e.g. ¼) and then increases the multiplier value only if the corresponding PWopt value is not within the range of programmable pulse widths. If none of the voltage multipliers has a corresponding PWopt value within the range of programmable pulse widths (i.e. none of the voltage multipliers is deemed “operable” for that vector), then the vector is rejected and others are instead analyzed. Assuming however that a lowest operable voltage multiplier is found for a selected vector, the system then determines the pulse charge value (Qbat_opt) corresponding to the PWopt value and the lowest operable voltage multiplier value (Mutt). This may be achieved by first determining the impedance (R) of the selected vector and then using:

$${Q}_{bat\_opt} = Mult \cdot \frac{{V}_{stim} \cdot {PW}_{opt}}{R}$$

wherein Vstim is the lowest operable multiplier (Mutt) times the battery voltage (Vbat) for the device.

The foregoing exemplary procedure may then be repeated for other vectors within a set of programmable vectors to identify the particular vector providing the lowest overall pulse charge value. This particular vector is selected for delivering stimulation to the patient since it consumes the least amount of charge per pulse while still achieving the corresponding capture threshold. Safety margins or safety factors are preferably employed in the analysis, such as by applying the Margin value as shown in the equations above. Note that the safety factor may be set lower if the device is equipped for capture verification. If a safety factor is used, it may be adjusted depending upon time since implant with a generally higher safety factor used during the post-implant acute phase and a generally lower value used during the subsequent chronic implant phase. Note also that if none of the vectors is found to have an operable multiplier value, suitable warning signals are generated for the patient or clinician and the pulse width and pulse voltage values are set to their highest programmable values in an attempt to achieve capture. In some examples, the system selects only among vectors capable of concurrent anodal and cathodal stimulation. Note also that if a selected vector results in unwanted phrenic stimulation, then another vector that does not trigger phrenic stimulation is instead selected. The system may also take into consideration interventricular conduction time delays (or other factors) when selecting vectors for stimulation.

Depending upon the particular implementation, the procedures may be performed by the implanted device or by an external system based on measured values transmitted from the implanted device (such as capture thresholds, impedance values, etc.), or some steps may be performed by the implanted device and others by the external system. In some embodiments, the foregoing assessment procedures are implemented as a “one button” energy optimization procedure using a device programmer, which automatically performs the assessment on behalf of a clinician to automatically identify the particular vector (or vector combination) that achieves the lowest pulse charge value without triggering phrenic stimulation. The selected vector and the corresponding pulse charge value are displayed for the clinician. Additionally or alternatively, the pulse charge value is converted into one or more of: a relative vector efficiency; a charge drawn from battery per pulse value; an energy from battery per pulse value; a microampere value of battery current drain associated with pacing; an incremental increase in current drain value; and an incremental expected decrease in longevity value, as these values might be helpful to the clinician. Note that the current drain corresponding to the lowest overall pulse charge value may be referred to herein as the “true absolute minimum battery current drain,” though it should be understood that various practical considerations may affect what constitutes the absolute minimum current drain within a particular device, such as nonlinearities in electronics, etc.

Within the examples described herein, the implantable device is a CRMD using a quad-pole left ventricular (LV) lead along with, at least, a right ventricular (RV) lead having an RV coil providing at least ten programmable stimulation vectors. However, aspects of the invention, are generally applicable to systems having other multi-pole LV leads or to systems having multi-pole RV leads and/or multi-pole right atrial (RA) leads, as well as to implantable devices besides CRMDs, such as devices for stimulating the spine or other organs.

### BRIEF DESCRIPTION OF THE DRAWINGS

Features and advantages of the described implementations can be more readily understood by reference to the following description taken in conjunction with the accompanying drawings.

FIG. 1 illustrates components of an implantable medical system having a CRMD equipped for voltage multiplier-based vector selection and pacing optimization;

FIG. 2 illustrates a multi-pole LV lead and various exemplary vectors for use with the system of FIG. 1;

FIG. 3 summarizes an exemplary voltage multiplier-based technique for optimizing current drain performed by the implantable system of FIGS. 1 and 2 or by an external system in communication therewith;

FIG. 4 provides a graph illustrating voltage strength duration curves exploited by the method of FIG. 3 for an example where a voltage divider can be used to reduce the charge needed per pulse;

FIG. 5 provides another graph of voltage strength duration curves similar to FIG. 4 but for an example where a voltage divider cannot be used to reduce the charge needed per pulse;

FIG. 6 illustrates an exemplary implementation of the method of FIG. 3 for determining a lowest operable voltage multiplier for a selected vector;

FIG. 7 illustrates an exemplary implementation of the method of FIG. 3 for selecting a particular vector for stimulation based on the lowest operable voltage multiplier and other factors;

FIG. 8 illustrates a exemplary method for use with the technique of FIG. 6 for determining rheobase and chronaxie for a selected vector;

FIG. 9 illustrates a exemplary method for use with the technique of FIG. 6 for determining PWopt for a selected vector and then for determining the lowest operable voltage multiplier;

FIG. 10 illustrates an exemplary display screen generated using the programmer of FIG. 1 for activating various of the techniques of FIGS. 3-9;

FIG. 11 is a simplified, partly cutaway view, illustrating the device of FIG. 1 along with a set of leads implanted into the heart of the patient;

FIG. 12 is a functional block diagram of the device of FIG. 11, illustrating basic circuit elements that provide cardioversion, defibrillation and/or pacing stimulation in the heart an particularly illustrating on-board components for use with the optimization techniques of FIGS. 3-9; and

FIG. 13 is a functional block diagram illustrating components of the external programmer of FIG. 1, particularly illustrating programmer-based components for controlling the optimization techniques of FIGS. 3-9.

### DETAILED DESCRIPTION OF THE PREFERRED EMBODIMENTS

The following description includes the best mode presently contemplated for practicing the invention. This description is not to be taken in a limiting sense but is made merely to describe general principles of the invention. The scope of the invention should be ascertained with reference to the issued claims. In the description of the invention that follows, like numerals or reference designators will be used to refer to like parts or elements throughout.

### Overview of Implantable Systems and Methods

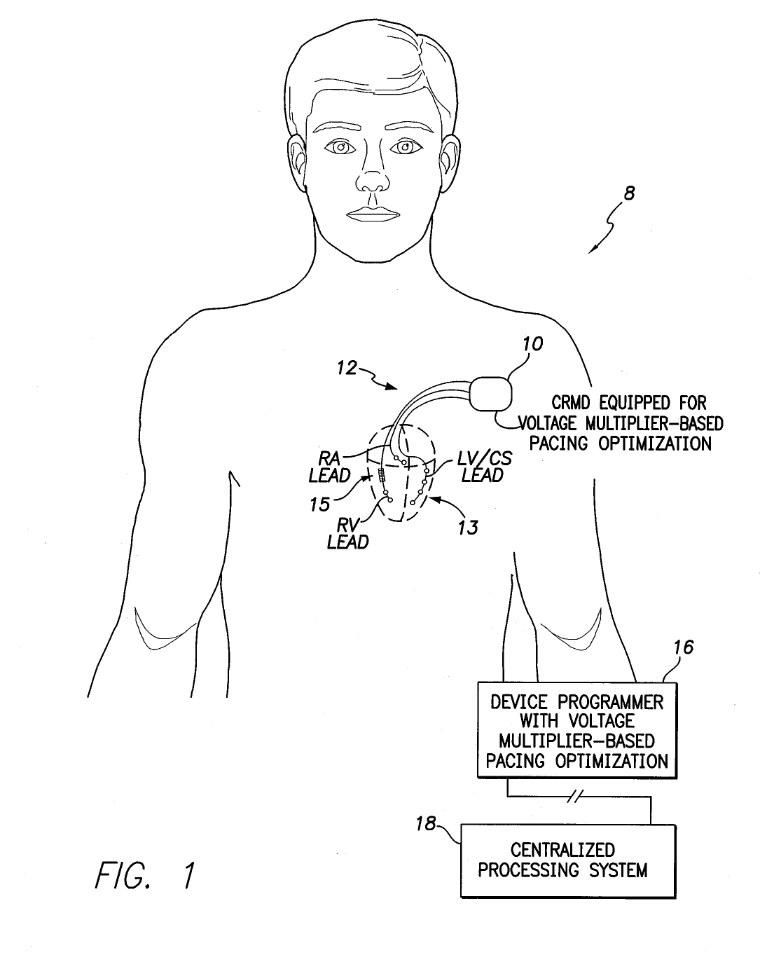

FIG. 1 illustrates an implantable medical system 8 equipped for voltage multiplier-based pacing optimization, operable alone or in conjunction with an external programmer device. In this example, the implantable medical system 8 includes a CRMD 10 (which may be a pacemaker, ICD, CRT or other suitable device) equipped with a set of cardiac leads 12 implanted on or within the heart of the patient, including a multi-pole LV lead implanted via the coronary sinus (CS). In FIG. 1, a stylized representation of the leads is provided. More accurate illustrations of the leads are provided within the other figures. To illustrate the multi-pole configuration of the LV lead, a set of electrodes 13 is shown. In the examples described herein, a quad-pole lead is employed (such as the Quartet™ lead provided by St Jude Medical.) Other suitable leads may instead be employed, including leads with more or fewer electrodes. Also, as shown, an exemplary RV lead is provided that includes an RV tip/ring electrode pair and an RV coil 15. An RA lead is also shown, which includes an RA tip/ring pair. Other electrodes of various sizes and shapes may be additionally or alternatively provided, such as coil electrodes mounted in the RA or in the CS near the left atrium (LA.) Where appropriate, the various leads can be intravenous, pericardial, endocardial or “leadless” pacing devices.

In some implementations, the CRMD itself performs the voltage multiplier-based pacing optimization based on parameters sensed or measured using its leads. In other implementations, the device transmits pertinent signals, data or other parameters to an external system such as device programmer 16 that performs the optimization procedure under the supervision of a clinician or other user. In some implementations, the procedure is implemented as a “one button” process to minimize any burden on the clinician. Note that other external systems might instead be employed to perform or control the optimization procedure, such as bedside monitors or the like. In some embodiments, the external system is directly networked with a centralized computing system 18, such as the HouseCall™ system or the Merlin@home/Merlin.Net systems of St. Jude Medical, which can perform at least some of the processing.

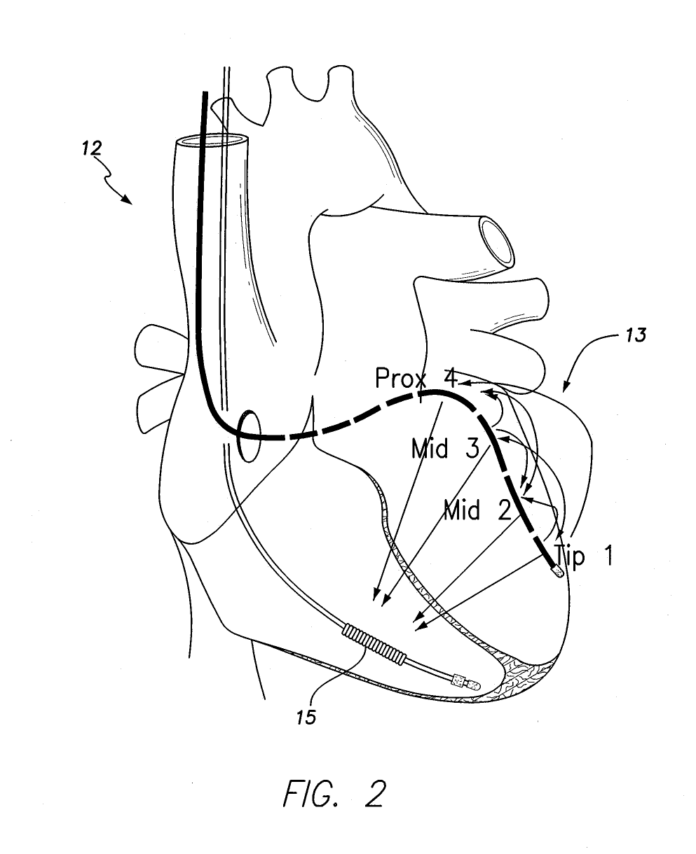

FIG. 2 provides another stylized illustration of the heart of a patient showing the RV and LV leads of lead system 12 in greater detail and, in particular, showing the four LV electrodes of the exemplary quad-pole LV lead. The LV electrodes are denoted—from distal LV to proximal LV—as: Tip 1 (or T1), Mid 2 (or M2), Mid 3 (or M3), and Prox 4 (or P4.) The figure also shows various pacing/sensing vectors between the LV electrodes and RV coil electrode 15 and further illustrates various LV interelectrode sensing vectors among the electrodes of the LV lead. A total of ten vectors are shown. Note that the particular locations of the implanted components shown in FIGS. 1 and 2 are merely illustrative and might not necessarily correspond to actual implant locations. Also, although the descriptions herein use the Quartet™ lead as an exemplary component, it should be understood that any suitable LV lead could instead be used. The ten vectors of FIG. 2 are also listed in Table I, which also identifies the cathode and anode for each vector.

TABLE I

| Vector | Description | Cathode | Anode |
|---|---|---|---|
| Vector 1 | Distal 1 to Mid 2 | Distal 1 | Mid 2 |
| Vector 2 | Distal 1 to Proximal 4 | Distal 1 | Proximal 4 |
| Vector 3 | Distal 1 to RV Coil | Distal 1 | RV Coil |
| Vector 4 | Mid 2 to Proximal 4 | Mid 2 | Proximal 4 |
| Vector 5 | Mid 2 to RV Coil | Mid 2 | RV Coil |
| Vector 6 | Mid 3 to Mid 2 | Mid 3 | Mid 2 |
| Vector 7 | Mid 3 to Proximal 4 | Mid 3 | Proximal 4 |
| Vector 8 | Mid 3 to RV Coil | Mid 3 | RV Coil |
| Vector 9 | Proximal 4 to Mid 2 | Proximal 4 | Mid 2 |
| Vector 10 | Proximal 4 to RV Coil | Proximal 4 | RV Coil |

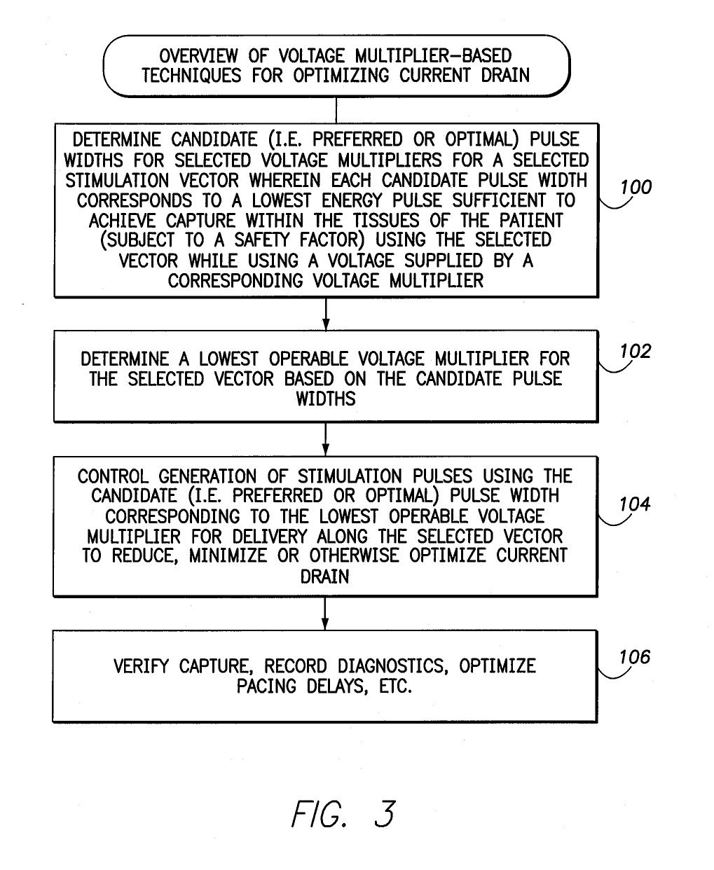

FIG. 3 broadly summarizes techniques exploited by the system of FIG. 1 (or other suitably-equipped systems) for optimizing pulse width, pacing voltage and vector selection based in part on voltage multiplier values. As noted, depending upon the capabilities of the device itself, the optimization techniques may be performed by the implanted device itself, an external system, or some combination thereof. Beginning at step 100, the system determines candidate (i.e. preferred or optimal) pulse widths for selected voltage multipliers for a selected stimulation vector. The implantable device may be equipped, for example, with a voltage halver, a voltage quarterer, a voltage doubler and a voltage tripler, thereby allowing for programmable selection of a particular multiplier. (Note that using a multiplier/divider is not the same as using an op-amp to step up or step down the voltage.) Each candidate pulse width (PWopt) corresponds to a lowest energy pulse sufficient to achieve capture within the tissues of the patient (subject to an optional safety margin factor) using the selected vector while using a voltage supplied by a corresponding selected voltage multiplier.

At step 102, the system determines a lowest operable voltage multiplier for the selected vector based on the candidate pulse widths. The lowest operable voltage multiplier represents the lowest multiplier sufficient to provide enough voltage to generate a pulse at the candidate pulse width (PWopt), at least within a range of programmable pulse widths supported by the device, such as range extending from 0.05 ms to 1.0 ms, inclusive. At step 104, the system then controls the generation of stimulation pulses using the candidate (i.e. preferred or optimal) pulse width corresponding to the lowest operable voltage multiplier for delivery along the selected vector to reduce, minimize or otherwise optimize current drain. At step 106, the implanted device verifies capture (if so programmed), records diagnostics, etc., as well as performing other pacing/sensing/shocking functions depending upon the capabilities and programming of the device.

The stimulation may include, e.g., biventricular pacing therapy or CRT. CRT and related therapies are discussed in, for example, U.S. Pat. No. 6,643,546 to Mathis et al., entitled “Multi-Electrode Apparatus and Method for Treatment of Congestive Heart Failure”; U.S. Pat. No. 6,628,988 to Kramer et al., entitled “Apparatus and Method for Reversal of Myocardial Remodeling with Electrical Stimulation”; and U.S. Pat. No. 6,512,952 to Stahmann et al., entitled “Method and Apparatus for Maintaining Synchronized Pacing”. See, also, U.S. Pat. No. 8,301,246 of Park et al., entitled “System and Method for Improving CRT Response and Identifying Potential Non-Responders to CRT Therapy”; and U.S. Pat. No. 7,653,436 of Schecter, entitled “Global Cardiac Performance.” See, also, U.S. Pat. No. 8,447,400 of More et al., entitled “Systems and Methods for Use by an Implantable Medical Device for Controlling Multi-Site CRT Pacing in the Presence of Atrial Tachycardia”; and U.S. Pat. No. 8,332,033 of Reed et al., entitled “Systems and Methods for use by an Implantable Medical Device for Controlling Multi-Site CR Pacing in the presence of Atrial Tachycardia.”

Hence, systems and methods are provided for optimizing pacing based, at least in part, on the lowest operable voltage multipliers associated with various stimulation vectors so as to reduce, minimize or otherwise optimize current drain. These techniques will now be described in more detail with reference to various exemplary embodiments.

### Illustrative Systems and Methods

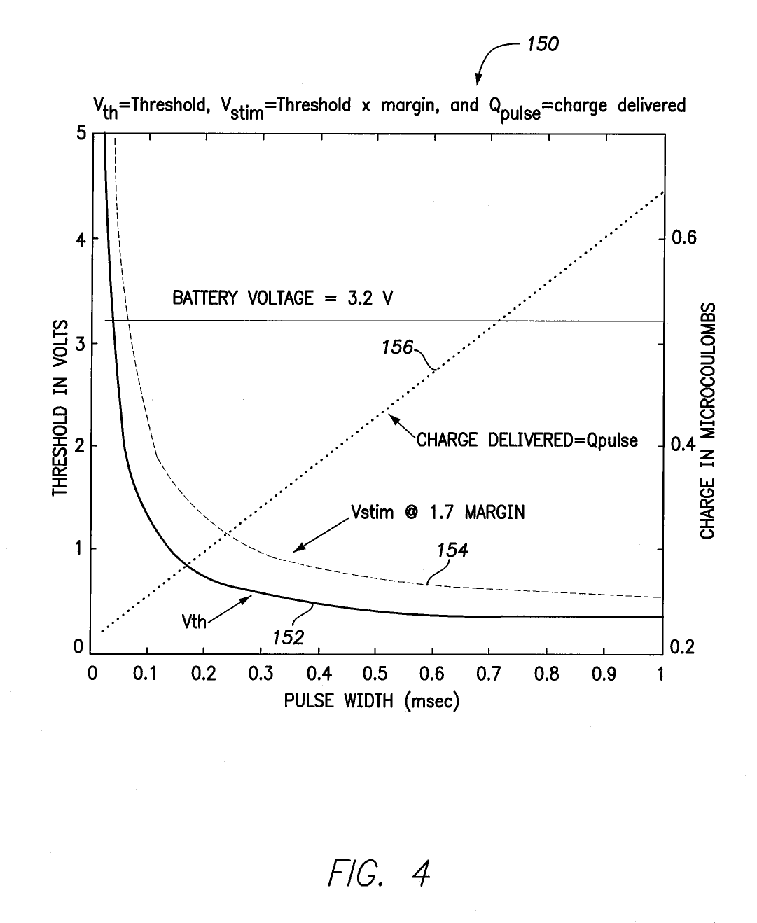

With reference to FIGS. 4-10, exemplary embodiments will be described in detail. Various technical and practical issues pertaining to battery drain within an implantable medical device are presented first. Energy drain from a battery associated with pacing is directly related to the current consumed by each pacing pulse. The energy delivered from the battery is: Energy=V_{bat} Q_{bat}. V_{bat} is the battery voltage in joules/coulomb and Q_{bat} is the charge delivered in coulombs. A one amp-hour lithium battery, typical capacity of a pacemaker battery, will deliver about 3600 coulombs at about 3.2 V or 11,520 joules. Battery current drain directly relates to the longevity of devices. For example, stimulating at 0.8 volts for 0.5 ms versus 2.5 volts at 0.5 ms at 60 pulses per minute will decrease device longevity of some exemplary devices by thirteen months. Minimizing battery current drain, however, is more complicated than simply identifying the pacing vector that has the lowest capture threshold. Charge delivered for each pacing pulse is directly proportional to the stimulation voltage and pulse width, and inversely related to pacing impedance:

$${Q}_{pulse} = \frac{{V}_{stim} \cdot PW}{R} . \qquad ( 1 )$$

Currently, the typical procedure for identifying the most energy-efficient pacing location is to perform a calculation for each of the available vectors, which can be a laborious task, particularly for devices with ten or more vectors. The calculation involves estimating the charge delivered with each pacing pulse (Qₚᵤₗₛₑ) with respect to pulse width (PW), impedance (R), and the stimulation pulse voltage (Vₛₜᵢₘ) where Vₛₜᵢₘ=Margin·Vₜₕ. “Margin” is the safety margin factor for stimulation, which is dependent on how frequently the threshold is updated as well as the relative risk of loss of capture. Typically, if the threshold is not measured often (e.g. only every six months or so), the safety margin can be set to 1.7 to 2.0 times the threshold. If thresholds are more frequently updated, the margin may be only 1.25 to 1.5 the measured threshold as is the case when a device automatically takes thresholds.

FIG. 4 presents a graph 150 showing a typical strength duration curve 152 that can be estimated by measuring the threshold at two pulse widths using the Lapicque equation:

$${V}_{th} = Rho \cdot ( 1 + \frac{Chron}{PW} ) . \qquad ( 2 )$$

Curve 152 is shown for typical values of rheobase (0.216 volts) and chronaxie (0.5 ms.) The pacing impedance is 500 ohms. An iso-safety margin line 154 is also plotted. The iso-safety margin is the set of Vₛₜᵢₘ points that may be chosen at any given pulse that provides the desired margin above the measured threshold account for variations in threshold. The iso-safety margin equation is:

$${V}_{stim} = Margin \cdot Rho \cdot ( 1 + \frac{Chron}{PW} ) . \qquad ( 3 )$$

The charge delivered with each stimulation pulse is also plotted 156 as a function of pulse width:

$${Q}_{pulse} = \frac{{V}_{stim} \cdot PW}{R} = \frac{1}{R} \cdot Margin \cdot Rho \cdot ( PW + Chron ) . \qquad ( 4 )$$

Note that the charge delivered is linearly related to pulse width. When pacing, the typical maximum voltage that may be delivered is the battery voltage that is used to fully charge the pacing capacitor (without using voltage multiplier circuitry to half, double, or triple the battery voltage). The curves of FIG. 4 show that the optimal stimulus duration, PWopt, is the pulse duration associated with the intersection of the battery voltage and Vstim. In this example, at the pulse width of 0.065 ms, the stimulus is 3.2 volts, while the charge delivered is 0.24 millicoulombs. This is the minimum charge required for pacing at the full battery voltage.

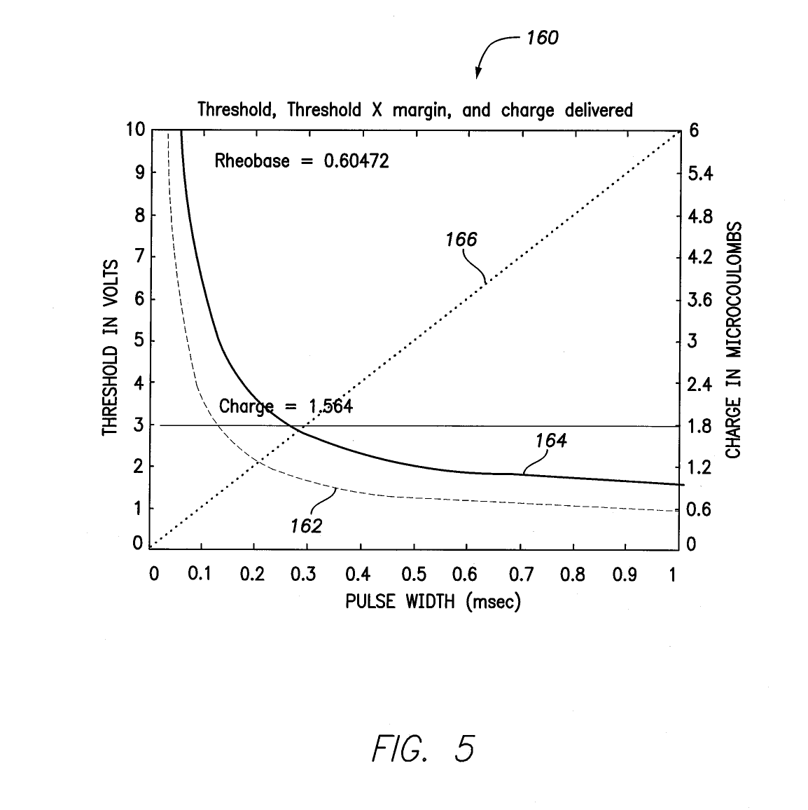

Further with regard to FIG. 4, it can be seen that the charge delivered decreases with decreasing pulse width. Therefore, the minimal pulse width at the maximum given practicable stimulation voltage at safety margin, Vₛₜᵢₘ=V_{bat}, draws the least charge from the battery. The battery is used to fully charge the pacing capacitor with the charge at 3.2 V and that charge in turn is delivered to the heart, Qₚᵤₗₛₑ. Note also that the minimum charge requirements may be achieved by charging the capacitor to the battery voltage and using the most narrow pulse width that achieves an adequate safety margin. Furthermore, the charge drawn from the battery may be further reduced by cutting the battery voltage in half using an efficient “voltage halver” circuit. For example, using the case shown in FIG. 4, when the stimulus voltage is cut in half to 1.6 V and consequently Vₛₜᵢₘ is set to 1.6 V, the charge required for stimulation is 0.28 millicoulombs, which is more charge than the charge required when stimulating with 3.2 V, which is 0.24 millicoulombs. However, when a halver circuit is used, the charge drawn from the battery is cut in half from 0.28 millicoulombs to 0.14 millicoulombs. This is lower than the charge drawn at 3.2 V, being 0.24 millicoulombs. Thus the voltage halver significantly reduces charge required for pacing by one half. Conversely, a voltage doubler (stimulating at 6.4 V) doubles the charge required for pacing, and a tripler circuit triples the charge drain at the battery. Reducing the voltage with a ¼ circuit would only require 0.1 microcoulombs from the battery. These effects/benefits achieved using voltage multipliers/dividers are generally known in the electrical arts. FIG. 5 provides another strength duration graph 160, including Vth 162, Vstim 164 and charge delivered 166. For this example, rheobase is higher (Rho=0.60472) and so a voltage divider cannot be used while still achieving the desired safety margin. That is, for this example, the lowest operable voltage multiplier value is 1.

Strength duration curves are also discussed in, e.g.: U.S. Pat. No. 5,697,956 to Bornzin, cited above, and in U.S. Pat. No. 7,574,259 to Pei et al., entitled “Capture threshold and Lead Condition Analysis” and U.S. Published Application 2009/0270938 of Pei et al., also entitled “Capture Threshold and Lead Condition Analysis.” See, also, U.S. Pat. No. 6,738,668 to Mouchawar et al., entitled “Implantable Cardiac Stimulation Device having a Capture Assurance System which Minimizes Battery Current Drain and Method for Operating the Same”; U.S. Pat. No. 6,615,082 to Mandell entitled “Method and Device for Optimally Altering Stimulation Energy to Maintain Capture of Cardiac Tissue” and U.S. Pat. No. 5,692,907 to Glassel et al., entitled “Interactive Cardiac Rhythm Simulator.” The Lapicque Equation is discussed in aforementioned patents to Mouchawar et al. (U.S. Pat. No. 6,738,668) and Mandell (U.S. Pat. No. 6,615,082) See, also, U.S. Pat. No. 6,549,806 to Kroll entitled “Implantable Dual Site Cardiac Stimulation Device having Independent Automatic Capture Capability” and U.S. Pat. No. 6,456,879 to Weinberg, entitled “Method and Device for Optimally Altering Stimulation Energy to Maintain Capture of Cardiac Tissue.” Rheobase and chronaxie are also discussed in U.S. Pat. No. 7,158,826 to Kroll et al.

Insofar as determining rheobase (Rho) and chronaxie (Chron) is concerned, these values can be estimated by performing two threshold measurements at two pulse widths: (Vₜₕ₁, PW₁); (Vₜₕ₂, PW₂). Substitution of the two points into equation (2) yields a system of two equations:

$${V}_{th 1} = Rho \cdot ( 1 + \frac{Chron}{{PW}_{1}} ) \\ {V}_{th 2} = Rho \cdot ( 1 + \frac{Chron}{{PW}_{2}} ) \qquad ( 5 )$$

Simultaneous solution of the system of equations (5) allows for calculating the chronaxie, Chron, and rheobase, Rho as shown in equations (6) and (7):

$$\begin{matrix}Rho = \frac{{V}_{th 2} - \frac{{PW}_{1}}{{PW}_{2}} \cdot {V}_{th 1}}{1 - \frac{{PW}_{1}}{{PW}_{2}}} \\ and & ( 6 ) \\ Chron = \frac{( {V}_{th 1} - Rho ) \cdot {PW}_{1}}{Rho} . & ( 7 )\end{matrix}$$

Selecting the appropriate optimal pulse width may be achieved by solving equation (3) for the PW yielding equation (8). Equation (8) should be solved using the lowest operable multiple of battery voltage, Mult, that achieves stimulation over the available range of pulse widths, i.e., 0.05 ms to 1 ms. In one example, the available multiples of pulse width, Mult, include Mult=½ when using a “halver circuit”, Mult=1 when using a full battery voltage circuit, Mult=2 when using a voltage “doubler circuit” and Mult=3 when using a “voltage tripler” circuit and hence Vstim=Vbat*Mult has four discrete output levels. If Vbat is 3.2, then for Mult=½, Vstim=1.6 V. For Mult=1, Vstim=3.2 V. For Mult=2, Vstim=6.4 V. For Mult=3, Vstim=9.6 V.

$${PW}_{opt} = \frac{Chron}{\frac{{V}_{stim}}{Margin \cdot Rho} - 1} {V}_{stim} . \qquad ( 8 )$$

Equation (9) is used to calculate the charge at the optimal pulse width after the pacing impedance, R, is measured.

$${Q}_{bat\_opt} = Mult \cdot \frac{{V}_{stim} \cdot {PW}_{opt}}{R} . \qquad ( 9 )$$

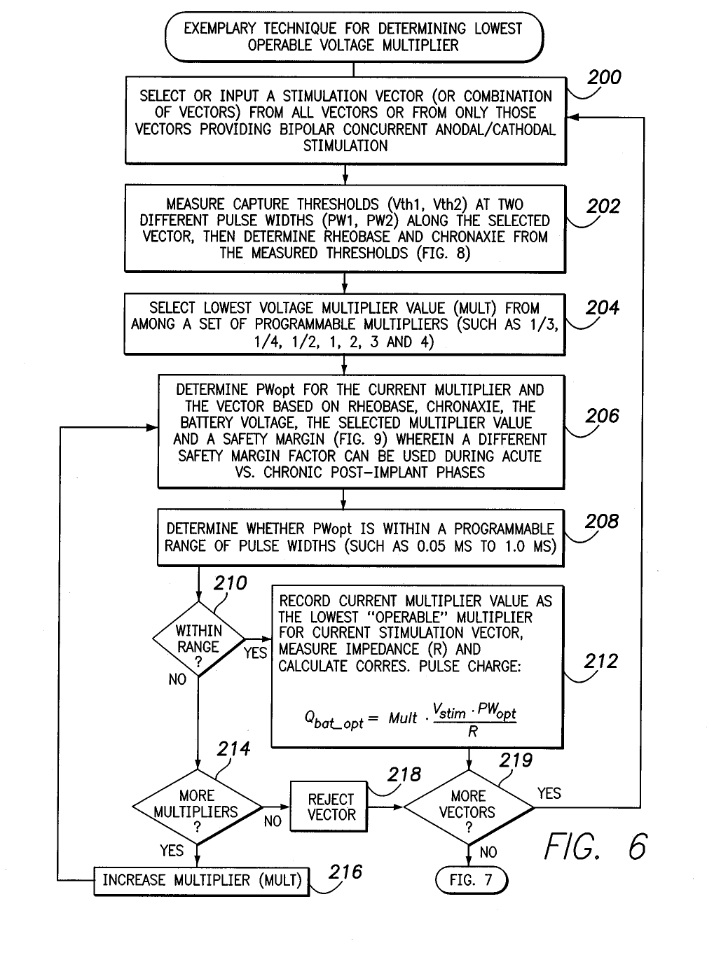

With the foregoing in mind, an exemplary technique for determining and exploiting the lowest operable voltage multiplier will now be described for an example where the system operates to select an optimal vector or combination of vectors based on current drain and other factors. Beginning at step 200 of FIG. 6, the system selects or inputs a stimulation vector or combination of vectors, where the selection can initially be made by a clinician and then input into the system. In the examples specifically described herein, the system selects/inputs one individual vector at a time where each vector corresponds to one anode/cathode pair. However, it should be understood that combinations of vectors could instead be selected. For example, stimulation might be delivered concurrently using Vectors 3 and 10 of Table I. For brevity, these descriptions will refer to the selection/processing of one vector at a time, with occasional reminders that combinations of vectors can instead be used.

Note that the system may select vectors at step 200 from among only those vectors providing concurrent anodal/cathodal stimulation. Systems and techniques exploiting anodic/cathodic capture are discussed in U.S. patent application Ser. No. 13/351,958, filed Jan. 17, 2012, of Hellman et al., entitled “Systems and Methods for Assessing and Exploiting Concurrent Cathodal and Anodal Capture using an Implantable Medical Device”; U.S. patent application Ser. No. 13/649,795, filed Oct. 11, 2012, of Bornzin, entitled “Systems and Methods for Packed Pacing using Bifurcated Pacing Pulses of Opposing Polarity Generated by an Implantable Medical Device”; and U.S. patent application Ser. No. 13/649,657, filed Oct. 11, 2012, of Bornzin et al., entitled “Systems and Methods for Postextrasystolic Potentiation using Anodic and Cathodic Pulses Generated by an Implantable Medical Device.” See, also, U.S. Pat. No. 8,380,307 to Lian et al., entitled “Switch Polarity Pacing to Improve Cardiac Resynchronization Therapy,” which discussed, inter alia, anodal and cathodal stimulation pulses of switchable polarity. For background regarding anodal capture, see, e.g. techniques described in U.S. Published Application 2010/0121396 of Gill et al., entitled “Enhanced Hemodynamics through Energy-Efficient Anodal Pacing” and U.S. patent application Ser. No. 11/961,720, filed Dec. 20, 2007, of Snell et al., entitled “Method and Apparatus with Anodal Capture Monitoring.”

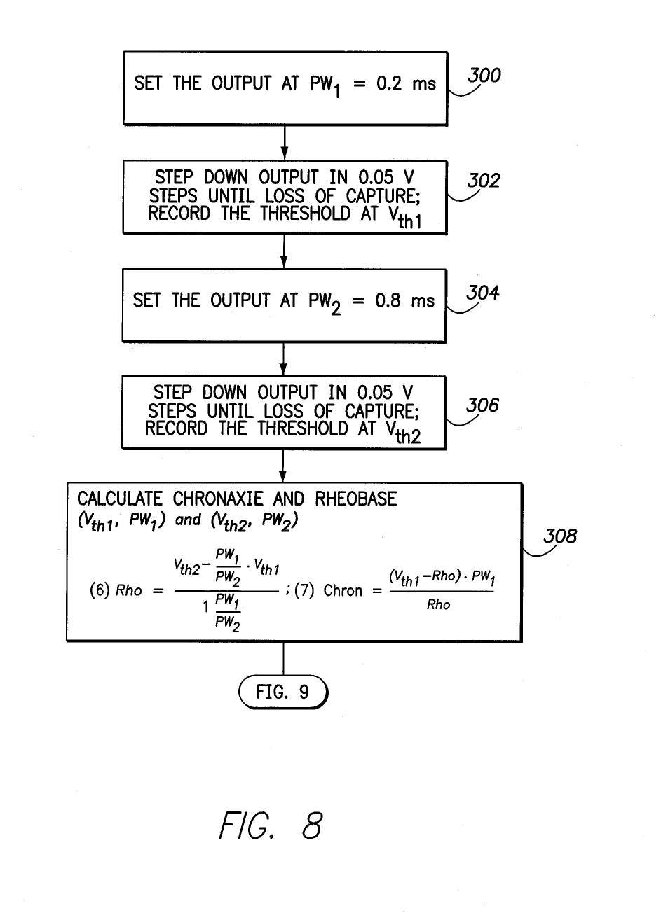

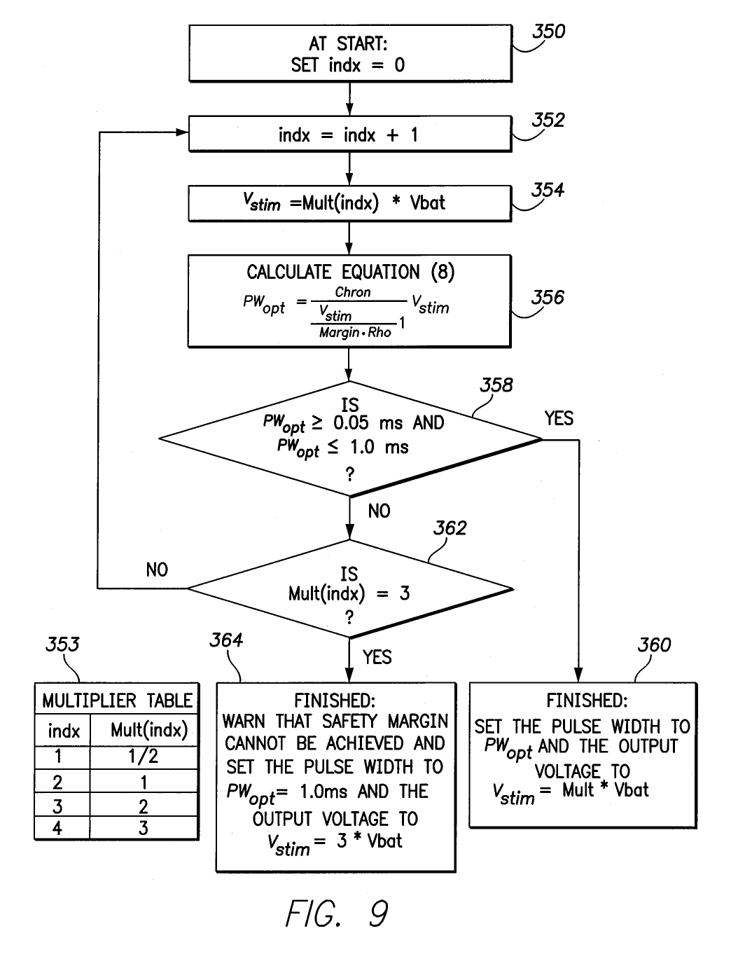

At step 202, the system measures capture thresholds (Vth1, Vth2) at two different pulse widths (PW1, PW2) along the selected vector, then determine rheobase (Rho) and chronaxie (Chron) from the measured thresholds, using techniques shown more fully in FIG. 8. At step 204, the system selects a lowest voltage multiplier value (Mutt) from among a set of programmable multiplier values (such as ⅓, ¼, ½, 1, 2, 3 and 4.) Then, for the selected vector and selected multiplier, the system at step 206 determines PWopt for the current multiplier and the vector based on rheobase, chronaxie, the battery voltage, the selected multiplier value and a safety margin, using techniques shown more fully in FIG. 9. Note that a different safety margin factor can be used during acute vs. chronic post-implant phases. This provides clinicians with an extra layer of control regarding battery drain especially during the 2-3 month acute post-implant phase where many clinicians program high output pacing during the lead maturation period and then forget to reprogram to lower output levels at subsequent follow-ups. One specific method for addressing this would be to utilize an “Energy Optimization” programmable safety margin of 2.5 times threshold for three months after the implant and then automatically reverting to a 1.7 times threshold setting thereafter.

As step 208, the system determines whether PWopt is within a programmable range of pulse widths (such as 0.05 ms to 1.0 ms.) If PWopt is within the range (step 210), then the current multiplier value is recorded at step 212 as the lowest “operable” multiplier for current stimulation vector. The device then measures values representative of impedance (R) along the selected vector and calculates the corresponding pulse charge using Eq. 9, or other suitable techniques. Where appropriate, the real component of impedance (i.e. resistance) is employed. In some examples, other related electrical parameters besides impedance might instead be measured, if appropriate, such as admittance, conductance or immittance, then converted as needed. Note that a particularly effective tri-phasic impedance detection pulse for use in measuring impedance is described in U.S. patent application Ser. No. 11/558,194 of Panescu et al., filed Nov. 9, 2006, entitled “Closed-Loop Adaptive Adjustment of Pacing Therapy based on Cardiogenic Impedance Signals Detected by an Implantable Medical Device.” See, also, techniques described in U.S. Published Application 2012/0035495 of Gutfinger et al., entitled “Systems and Methods for Exploiting Near-Field Impedance and Admittance for use with Implantable Medical Devices” and U.S. Published Application 2012/0035493 of Gutfinger et al., filed Aug. 9, 2010, entitled “Near Field-Based Systems and Methods for Assessing Impedance and Admittance for use with an Implantable Medical Device.” Insofar as calculating or determining the pulse charge, rather than using Eq. 9, the device could instead measure the amount of charge drawn from the capacitor or use a model of the capacitor to estimate charge.

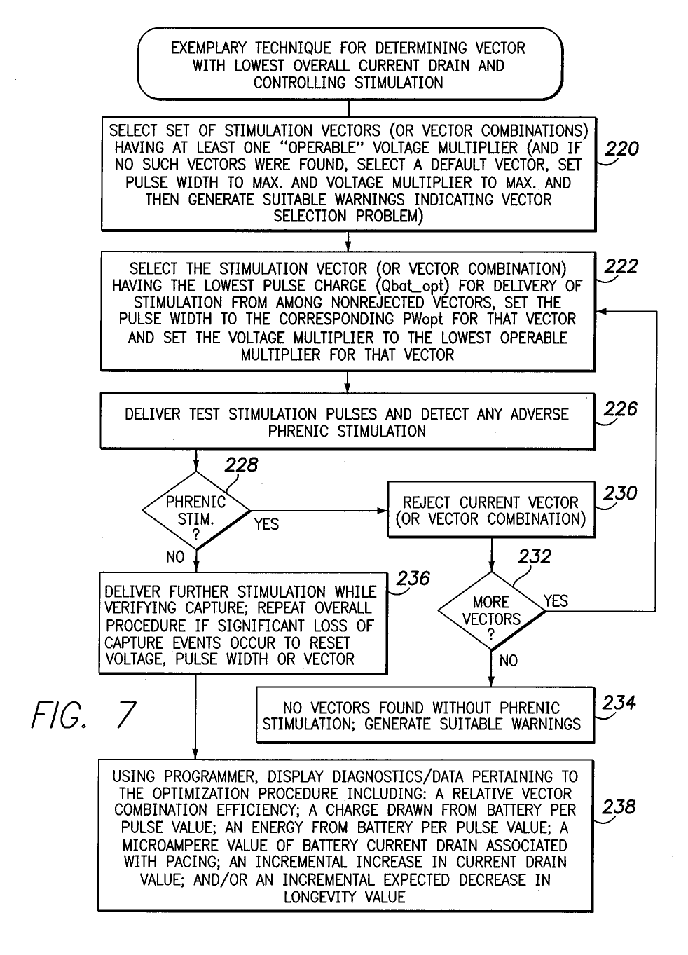

If the PWopt value determined at step 206 is not within the programmable range of pulse widths, then the currently selected multiplier is not deemed “operable” (at least for the selected vector). Accordingly, following step 210, the system verifies at step 214 that the Mult value can be increased (i.e. the currently selected Mult value is not already the highest available value) and the system then increases Mult at step 216. Thereafter steps 206 and 208 are repeated with the new higher Mult value to determine a new PWopt and compare it to the range of permissible pulse widths. In this manner, the Mult value is increased until either the corresponding voltage multiplier is found to be operable (step 212) or the vector is rejected at step 218 since no multiplier was found to be operable for that vector. In either case, assuming there are additional vectors to be analyzed, processing returns to step 200 via step 219, and the analysis procedure is repeated for another vector (or vector combination.) Once the last of the vectors has been analyzed, processing proceeds to FIG. 7 to determine the vector having the lowest current drain and control delivery of stimulation.

At step 220 of FIG. 7, the system selects the set of stimulation vectors (or vector combinations) having at least one “operable” voltage multiplier for further processing. Note that in the unlikely event that none of the vectors (or vector combinations) analyzed in FIG. 6 were found to have an operable multiplier, then suitable warnings are generated to alert the clinician and/or patient. The system may then set the pulse width to a maximum value and the voltage multiplier to its maximum value in an attempt to provide any needed therapeutic stimulation. If such a situation occurs, it may be because the safety margin is set to high, which the clinician can then adjust, or perhaps there is a problem with the leads or their positioning within the tissues of the patient. Assuming, however, that a set of vectors (or vector combinations) are found to have operable multipliers, the system at step 222 selects the stimulation vector (or vector combination) having the lowest pulse charge (Qbat_opt) for delivery of stimulation from among the “nonrejected” vectors. The system also sets the pulse width to the corresponding PWopt value for that vector and sets the voltage multiplier to the lowest operable multiplier for that vector (or vector combination.)

At step 226, the implanted device then delivers test pulses based on the currently selected parameters and detects any adverse phrenic stimulation (i.e. diaphragmatic stimulation), which can occur if the pulses are too large in magnitude and/or the stimulation vector is too close to the phrenic nerves. Phrenic stimulation is discussed, for example, in U.S. Pat. No. 7,299,093 to Zhu et al., entitled “Method and Apparatus for Avoidance of Phrenic Nerve Stimulation during Cardiac Pacing”, which describes a CRT device in which an accelerometer is used to detect phrenic or other skeletal muscle contraction associated with the output of a pacing pulse. See, also, U.S. Pat. No. 6,772,008 to Zhu. Alternatively, the clinician may simply monitor the patient to determine if phrenic stimulation is occurring (assuming the procedure of FIG. 7 is being performed under clinician supervision.) If at step 228, phrenic stimulation is detected, the vector is rejected at step 230. Assuming additional vectors are available, the test procedure is repeated beginning at step 222 with the vector (or vector combination) having the next lowest Qbat_opt value. In the unlikely event that no more vectors are available at step 232 (i.e. all vectors have been rejected either for lacking an operable multiplier or for triggering phrenic stimulation), then suitable warnings are generated at step 234. Assuming however that at least one of the vectors (or vector combinations) passes the phrenic stimulation test, then at step 236 the implantable device is controlled to deliver further stimulation using the currently selected vector and its lowest operable multiplier while verifying capture (if so programmed). The overall procedure of FIGS. 6 and 7 can be repeated. This may be done either periodically (e.g. every few months) or on demand (such as if a significant number of loss of capture events (LOCs) are detected indicating the stimulation voltage may be set too low.)

Assuming the procedure is performed under clinician supervision using a device programmer then, at step 238, the programmer can be controlled to display diagnostics/data pertaining to the optimization procedure including: a relative vector combination efficiency; a charge drawn from battery per pulse value; an energy from battery per pulse value; a microampere value of battery current drain associated with pacing; an incremental increase in current drain value; and/or an incremental expected decrease in longevity value. Although step 238 is shown as the last step in the method of FIGS. 6 and 7, it should be understood that this data, or other data, might be displayed at other times during the operation to the procedure, depending upon the programming of the system. See, also, U.S. Pat. No. 8,401,646 of Stadler, et al., issued Mar. 19, 2013.

Turning now to FIGS. 8 and 9, additional exemplary processing details are described with reference to an example where only a single vector is analyzed. It should be understood that these techniques may also be applied when analyzing a set of vectors (or vector combinations.) In the example of FIG. 8, at step 300 the system begins a procedure for determining Rho and Chron by setting the output at PW1=0.2 ms. At step 302, the system steps down the output in 0.05 V steps until loss of capture occurs and then records the corresponding threshold as Vth1. Next, at step 304, the system resets the output to PW2=0.8 ms and then, at step 306, steps down the output in 0.05 V steps until loss of capture occurs, at which point the corresponding threshold is recorded as Vth2. These are, of course, merely exemplary values and different values can instead be used depending upon the particular device. At step 308, Eqs. (6) and (7) are used to calculate Rho and Chron.

Next, in the example, of FIG. 9, the system begins the procedure of determining the lowest operable Mult value by setting an index value (indx) to 0 at step 350. At step 352, the system increments the index and at step 354 sets Vstim=Mult(indx)*Vbat. Table 353 of FIG. 9 shows index values and corresponding multiplier values for one particular example. At step 356, the system determines PWopt from Eq. 8 and, at step 358, determines whether the current value of PWopt is within the range of programmable PW values. Assuming PWopt is within that range, i.e. Mult(indx) is “operable”, the pulse width is then set at step 360 to the current value of PWopt and the output voltage is set to Vstim=Mult*Vbat. If however the latest value of PWopt is not within the programmable range (step 358), the index value is again incremented at step 352 to examine the next multiplier value. If no further multipliers are available, as determined at step 362, then step 364 is performed to warn that the safety margin cannot be achieved using any of the multipliers and the pulse width is set to its maximum value (e.g. 1.0 ms) and Vstim is set to Vstim=3*Vbat. (This example assumes that the device does not have any voltage multipliers beyond a tripler and that no other vectors can be selected.)

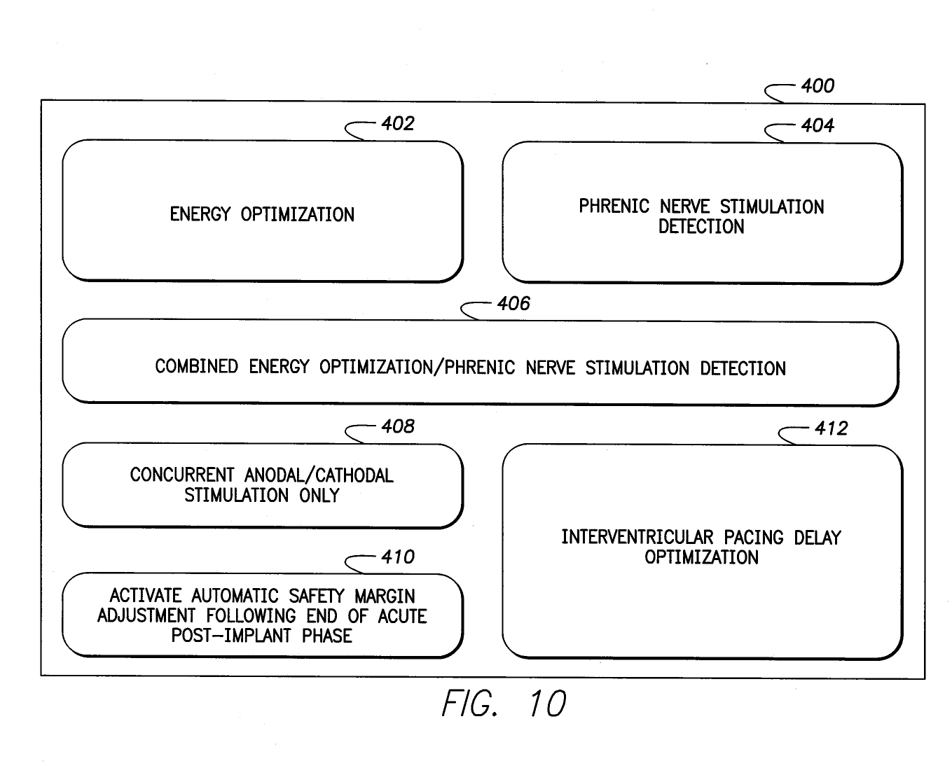

FIG. 10 illustrates commands presented on a display screen 400 of a device programmer (or other external system) for controlling or activating the various procedures described above. Briefly, an Energy Optimization “button” or command 402 activates an automatic search for the vector/pulse-width/voltage-multiplier combination having the lowest overall charge per pulse (without phrenic nerve stimulation detection.) That is, command 402 activates a procedure for automatically estimating the most energy efficient setting for each of the ten aforementioned stimulation vectors, which may be referred to as VectSelect™ pacing combinations or configurations where VectSelect™ is a trademark of Pacesetter Inc. The procedure automatically tests all ten pacing configurations and reports to the clinician the most efficient pacing combination and allows the clinician to choose the desired electrode combination. This allows clinicians to easily and automatically cycle through all ten exemplary vectors (while preferably using the detection of LV evoked responses to confirm capture) and should take approximately two minutes to complete. In order to confirm capture, the evoked responses may be detected on an adjacent electrode pair or on the actual electrode performing the pacing. The relative efficiency of a pacing combination may be quantified and reported (via printout or display) using a number of different metrics: charge drawn from battery per pulse; energy from battery per pulse; microamperes of battery current drain associated with pacing; incremental increases in current drain; and/or incremental expected decreases in longevity reported in months. For instance, the best electrode configuration current drain may be subtracted from the other electrode combinations to provide the incremental relative increase in current drain. When reported, the various electrode combinations can be displayed as a sorted list from the least efficient to the most efficient electrode combinations. Note also that less efficient Energy Optimization procedures or algorithms might be selected such as constant pulse width algorithms that do not consider the opportunity to use the narrowest pulse widths at the lowest multiple of battery voltage.

A Phrenic Nerve Stimulation Detection “button” or command 404 activates an automatic search to detect phrenic nerve stimulation. That is, this option triggers the system to automatically cycle through the electrode combinations while using stimulus amplitudes estimated during the Energy Optimization test. The system detects which electrode combination(s) stimulate the phrenic nerve using, e.g., an impedance plethysmograph system built into the implanted device or by some other method (e.g. chest accelerometer). The process may also be performed in a semiautomatic means. In this case, the device will cycle through the electrode combinations while the clinician (or other observer) notes on the programmer which combination of electrodes causes phrenic stimulation. The vector combinations that cause phrenic stimulation may then be eliminated from the list of programmable options. Note that Phrenic Nerve Stimulation Detection does not need to be performed if the clinician has already marked or identified certain electrode combinations in the device via the programmer as inappropriate because of phrenic nerve stimulation. Another option for the clinician is to interleave the Energy Optimization procedure with the steps of Phrenic Nerve Stimulation Detection procedure, which is activated via command 406. In this case (shown above in FIG. 7), the thresholds are measured and the most efficient setting is established for several cardiac cycles to determine if phrenic nerve stimulation present. For example, once the thresholds are measured and the most energy efficient setting is established, stimulation proceeds for several additional cardiac cycles to determine if phrenic nerve stimulation present. The programmer then proceeds to the next of the ten vector combinations. The process can also be performed at a safety margin greater than the programmed safety margin, e.g. 2× threshold or 3× threshold.

Command 408 activates procedures directed to measuring thresholds, assessing energy efficiency, etc., when performing simultaneous or concurrent anodal and cathodal stimulation on the bipolar combinations (e.g. Vectors 1, 2, 4, 6, 7, and 9 of Table I.) Simultaneous dual site ventricular pacing is advantageous because it can decrease activation times and increase cardiac performance. Since dual site simultaneous pacing requires more energy, it is important to choose the stimulus and the polarity of the stimulus that provides the highest energy efficiency. Therefore, the procedure tests with the primary polarity and switches to reversed polarity in order to determine the highest efficiency. Clinicians that prefer simultaneous dual site pacing may thereby select command 408 to only test the available bipolar vectors. Dual site capture is apparent when the bipolar sensed evoked response disappears, as is well known in the evoked response sensing art. This is because the simultaneous electrical depolarizations at the electrode tissue interface cancel one another. For example when pacing on Vector 1 and sensing between the electrode pair of Vector 1, there will be three characteristic electrograms: 1) Low output will display no capture on either electrode and thus no evoked response; 2) when cathodal stimulation commences, there will be a single evoked response from the cathode, and finally 3) when both the anode and cathode capture there will be almost no evoked response sensed because the evoked responses from each electrode are very similar and track one another. See, e.g., U.S. patent application Ser. No. 13/351,958, cited above.

Command 410 activates automatic safety margin adjustment following end of acute post-implant phase. This provides clinicians with an extra layer of control regarding battery drain especially during the 2-3 month acute post-implant phase where many clinicians program high output pacing during the lead maturation period and then forget to reprogram to lower output levels at subsequent follow-ups. One example utilizes an Energy Optimization programmable safety margin of 2.5× threshold for 3 months after the implant and then automatically reverts to a 1.7× threshold setting at the optimal pacing configuration thereafter. Command 412 activates interventricular pacing delay optimization based on the selected vector (or vector combinations) wherein, for example, pacing pulses are delivered to the LV and the delay to the RV is measured. The vector having the longest delay is then selected for biventricular pacing, assuming the corresponding current drain is acceptable (as determined via energy optimization.) That is, the choice of pacing vector depends not only on the current drain associated with PWopt but on the length of the interventricular delay or other delays.

Techniques for determining preferred or optimal interventricular (VV) delays or other stimulation delays are discussed, e.g., in U.S. Pat. No. 7,248,925 to Bruhns et al., entitled “System and Method for Determining Optimal Atrioventricular Delay based on Intrinsic Conduction Delays” and in at least some of the following patent documents: U.S. Published Application 2005/0125041, entitled “Methods for Ventricular Pacing”; U.S. patent application Ser. No. 10/974,123, filed Oct. 26, 2004; U.S. Pat. No. 7,590,446; U.S. patent application Ser. No. 10/980,140, filed Nov. 1, 2004; U.S. patent application Ser. No. 11/129,540, filed May 13, 2005; U.S. patent application Ser. No. 11/952,743, filed Dec. 7, 2007. See, also, U.S. Published Application 2010/0145405, entitled “Systems and Methods for Controlling Ventricular Pacing in Patients with Long Intra-Atrial Conduction Delays”, U.S. Published Application 2009/0299423, entitled “Systems and Methods for determining Intra-Atrial Conduction Delays using Multi-Pole Left Ventricular Pacing/Sensing Leads”, and U.S. Pat. No. 8,396,551, entitled “Systems and Methods for Optimizing Ventricular Pacing Delays During Atrial Fibrillation.”

See, also, the following patents and patent applications that set forth various systems and methods for determining and/or adjusting AV/VV pacing delays so as to help maintain the pacing delays at preferred or optimal values: U.S. Pat. No. 7,590,446; U.S. Published Application 2009/0299423; U.S. patent application Ser. No. 11/952,743, filed Dec. 7, 2007; U.S. Published Application 2010/0145405; U.S. Pat. No. 8,265,755; U.S. Published Application 2011/0022112; and U.S. Pat. No. 8,145,311 of Min et al., entitled “Systems and Methods for Determining Optimal Electrode Pairs for use in Biventricular Pacing using Multi-Pole Ventricular Leads.”

What have been described are various techniques for expediting pacing vector selection and to reduce or minimize current drain by selecting optimal pulse widths, voltage and vectors. It should be understood that these optimal parameters are not necessarily absolutely optimal in any rigorous mathematical sense. As can be appreciated, what constitutes “optimal” depends on the criteria used for judging the resulting performance, which can be subjective in the minds of some clinicians. Accordingly, the parameters identified herein are at least “preferred” parameters. Clinicians may choose to adjust or alter the selection via device programming for particular patients, at their discretion. Note also that, in the examples described herein, the multi-pole ventricular lead is an LV lead but aspects of the invention are applicable to multi-pole RV leads as well. Indeed, at least some of the techniques described herein are generally applicable to implementations wherein both the LV and RV have multi-pole leads. At least some of the techniques might also be applicable to multi-pole atrial leads implanted on or in either the RA or the LA/CS.

Among other features, the systems and techniques described herein provide for: a) scanning through all available vector combinations to find the most efficient combinations based on estimated current drain from the battery; b) providing a metric of efficiency on a printout using longevity increase, energy requirements, charge or current requirements and rank orders the performance of vectors; c) providing for programming bipolar-only configurations to ensure dual site simultaneous stimulation if that is the physician preference; d) allowing for semi-automatic or fully automatic (sensor-based) testing of phrenic nerve stimulation using different pulse amplitudes for each test that are selected based on a programmable stimulation safety margin (not on an arbitrary large output, i.e. 5V @ 0.5 ms); e) allowing for time efficient interleaved Energy Optimization and Phrenic Nerve Stimulation testing for each vector combination rather than performing Energy Optimization steps and then performing Phrenic Nerve Stimulation testing sequentially; f) performing procedures automatically in the device as a programmable option to allow turning it “ON” in the field; and g) using a programmable higher safety margin during the lead maturation phase and revert to a lower, more energy efficient safety margin after lead maturation.

For the sake of completeness, an exemplary CRMD will now be described that includes components for performing the functions and steps already described, although other implantable medical devices may be equipped to exploit the techniques described herein.

### Exemplary CRMD

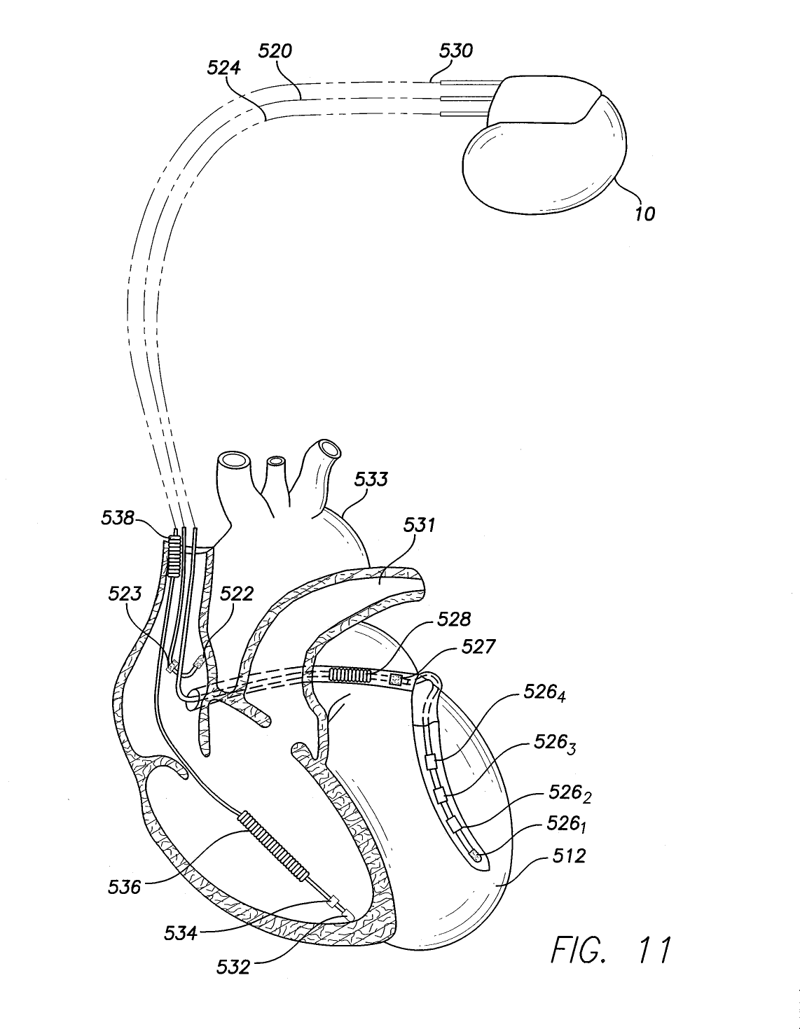

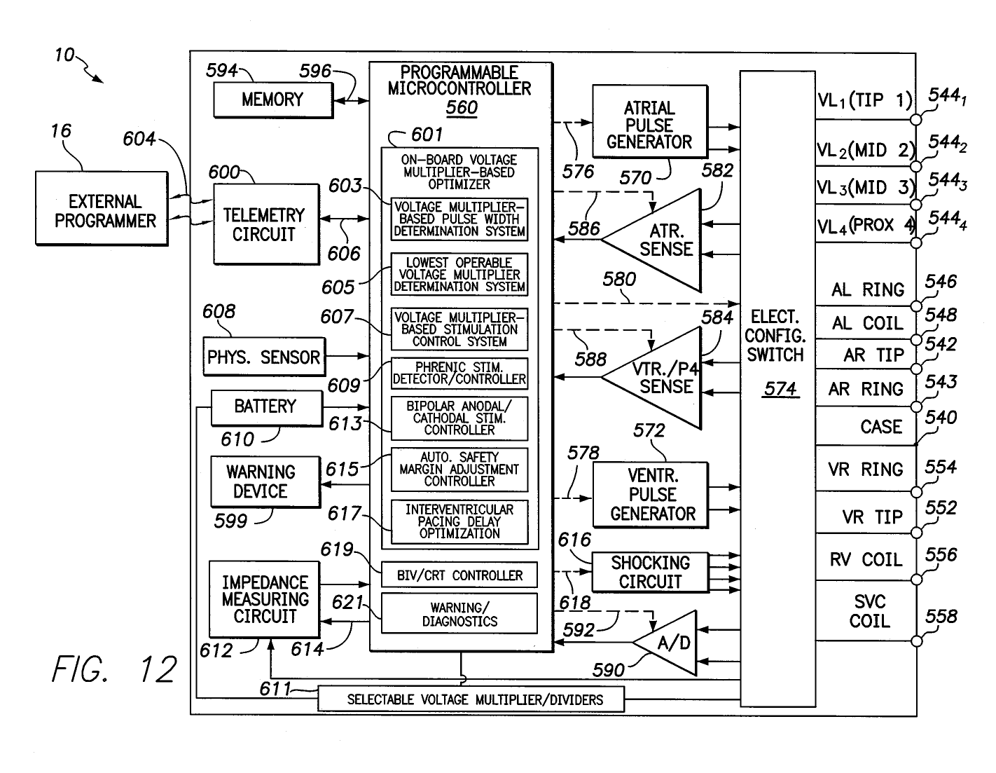

With reference to FIGS. 11 and 12, a description of an exemplary CRMD will now be provided. FIG. 11 provides a simplified block diagram of the CRMD, which is a dual-chamber stimulation device capable of treating both fast and slow arrhythmias with stimulation therapy, including cardioversion, defibrillation, and pacing stimulation, and also capable of selecting pacing vectors as discussed above. To provide right atrial chamber pacing stimulation and sensing, CRMD 10 is shown in electrical communication with a heart 512 by way of a right atrial lead 520 having an atrial tip electrode 522 and an atrial ring electrode 523 implanted in the atrial appendage. CRMD 10 is also in electrical communication with the heart by way of a right ventricular lead 530 having, in this embodiment, a ventricular tip electrode 532, a right ventricular ring electrode 534, a right ventricular (RV) coil electrode 536, and a superior vena cava (SVC) coil electrode 538. Typically, the right ventricular lead 530 is transvenously inserted into the heart so as to place the RV coil electrode 536 in the right ventricular apex, and the SVC coil electrode 538 in the superior vena cava. Accordingly, the right ventricular lead is capable of receiving cardiac signals, and delivering stimulation in the form of pacing and shock therapy to the right ventricle.

To sense left atrial and ventricular cardiac signals and to provide left chamber pacing therapy, CRMD 10 is coupled to a multi-pole LV lead 524 designed for placement in the “CS region” via the CS os for positioning a distal electrode adjacent to the left ventricle and/or additional electrode(s) adjacent to the left atrium. As used herein, the phrase “CS region” refers to the venous vasculature of the left ventricle, including any portion of the CS, great cardiac vein, left marginal vein, left posterior ventricular vein, middle cardiac vein, and/or small cardiac vein or any other cardiac vein accessible by the CS. Accordingly, an exemplary LV lead 524 is designed to receive atrial and ventricular cardiac signals and to deliver left ventricular pacing therapy using a set of four left ventricular electrodes 526₁ (Tip 1), 526₂ (Mid 2), 526₃ (Mid 3), and 526₄ (Prox 4), (thereby providing a quad-pole lead), left atrial pacing therapy using at least a left atrial ring electrode 527, and shocking therapy using at least a left atrial coil electrode 528. The 526₁ LV electrode may also be referred to as a “tip” or “distal” LV electrode. The 526₄ LV electrode may also be referred to as a “proximal” LV electrode. In other examples, more or fewer LV electrodes are provided. Although only three leads are shown in FIG. 11, it should also be understood that additional leads (with one or more pacing, sensing and/or shocking electrodes) might be used and/or additional electrodes might be provided on the leads already shown, such as additional electrodes on the RV lead. It is also noted that, on present commercially-available hardware, there is often no separate electrode 527. That is, the Prox 4 electrode 526₄ and the “left atrial ring electrode” 527 are the same. Both electrodes are shown for the sake of completeness and generality.

A simplified block diagram of internal components of CRMD 10 is shown in FIG. 12. While a particular CRMD is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation. The housing 540 for CRMD 10, shown schematically in FIG. 12, is often referred to as the “can”, “case” or “case electrode” and may be programmably selected to act as the return electrode for all “unipolar” modes. The housing 540 may further be used as a return electrode alone or in combination with one or more of the coil electrodes, 528, 536 and 538, for shocking purposes. The housing 540 further includes a connector (not shown) having a plurality of terminals, 542, 543, 544₁-544₄, 546, 548, 552, 554, 556 and 558 (shown schematically and, for convenience, the names of the electrodes to which they are connected are shown next to the terminals). As such, to achieve right atrial sensing and pacing, the connector includes at least a right atrial tip terminal (A_{R} TIP) 542 adapted for connection to the atrial tip electrode 522 and a right atrial ring (A_{R} RING) electrode 543 adapted for connection to right atrial ring electrode 523. To achieve left chamber sensing, pacing and shocking, the connector includes a left ventricular tip terminal (VL₁ TIP) 544₁ and additional LV electrode terminals 544₂-544₄ for the other LV electrodes of the LV lead.

The connector also includes a left atrial ring terminal (A_{L} RING) 546 and a left atrial shocking terminal (A_{L} COIL) 548, which are adapted for connection to the left atrial ring electrode 527 and the left atrial coil electrode 528, respectively. To support right chamber sensing, pacing and shocking, the connector further includes a right ventricular tip terminal (V_{R} TIP) 552, a right ventricular ring terminal (V_{R} RING) 554, a right ventricular shocking terminal (V_{R} COIL) 556, and an SVC shocking terminal (SVC COIL) 558, which are adapted for connection to the right ventricular tip electrode 532, right ventricular ring electrode 534, the V_{R} coil electrode 536, and the SVC coil electrode 538, respectively.

At the core of CRMD 10 is a programmable microcontroller 560, which controls the various modes of stimulation therapy. As is well known in the art, the microcontroller 560 (also referred to herein as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 560 includes the ability to process or monitor input signals (data) as controlled by a program code stored in a designated block of memory. The details of the design and operation of the microcontroller 560 are not critical to the invention. Rather, any suitable microcontroller 560 may be used that carries out the functions described herein. The use of microprocessor-based control circuits for performing timing and data analysis functions are well known in the art.

As shown in FIG. 12, an atrial pulse generator 570 and a ventricular pulse generator 572 generate pacing stimulation pulses for delivery by the right atrial lead 520, the right ventricular lead 530, and/or the LV lead 524 via an electrode configuration switch 574. It is understood that in order to provide stimulation therapy in each of the four chambers of the heart, the atrial and ventricular pulse generators, 570 and 572, may include dedicated, independent pulse generators, multiplexed pulse generators or shared pulse generators. The pulse generators, 570 and 572, are controlled by the microcontroller 560 via appropriate control signals, 576 and 578, respectively, to trigger or inhibit the stimulation pulses.

The microcontroller 560 further includes timing control circuitry (not separately shown) used to control the timing of such stimulation pulses (e.g., pacing rate, AV delay, atrial interconduction (inter-atrial) delay, or ventricular interconduction (V-V) delay, etc.) as well as to keep track of the timing of refractory periods, blanking intervals, noise detection windows, evoked response windows, alert intervals, marker channel timing, etc., which is well known in the art. Switch 574 includes a plurality of switches for connecting the desired electrodes to the appropriate I/O circuits, thereby providing complete electrode programmability. Accordingly, the switch 574, in response to a control signal 580 from the microcontroller 560, determines the polarity of the stimulation pulses (e.g., unipolar, bipolar, combipolar, etc.) by selectively closing the appropriate combination of switches (not shown) as is known in the art. The switch also switches among the various LV electrodes and the various pacing vectors that use the LV electrodes as cathodes.

Atrial sensing circuits 582 and ventricular sensing circuits 584 may also be selectively coupled to the right atrial lead 520, LV lead 524, and the right ventricular lead 530, through the switch 574 for detecting the presence of cardiac activity in each of the four chambers of the heart. Accordingly, the atrial (ATR. SENSE) and ventricular (VTR. SENSE) sensing circuits, 582 and 584, may include dedicated sense amplifiers, multiplexed amplifiers or shared amplifiers. The switch 574 determines the “sensing polarity” of the cardiac signal by selectively closing the appropriate switches, as is also known in the art. In this way, the clinician may program the sensing polarity independent of the stimulation polarity. Each sensing circuit, 582 and 584, preferably employs one or more low power, precision amplifiers with programmable gain and/or automatic gain control, bandpass filtering, and a threshold detection circuit, as known in the art, to selectively sense the cardiac signal of interest. The automatic gain control enables CRMD 10 to deal effectively with the difficult problem of sensing the low amplitude signal characteristics of atrial or ventricular fibrillation. The outputs of the atrial and ventricular sensing circuits, 582 and 584, are connected to the microcontroller 560 which, in turn, are able to trigger or inhibit the atrial and ventricular pulse generators, 570 and 572, respectively, in a demand fashion in response to the absence or presence of cardiac activity in the appropriate chambers of the heart.

For arrhythmia detection, CRMD 10 utilizes the atrial and ventricular sensing circuits, 582 and 584, to sense cardiac signals to determine whether a rhythm is physiologic or pathologic. As used in this section “sensing” is reserved for the noting of an electrical signal, and “detection” is the processing of these sensed signals and noting the presence of an arrhythmia. The timing intervals between sensed events (e.g., AS, VS, and depolarization signals associated with fibrillation which are sometimes referred to as “F-waves” or “Fib-waves”) are then classified by the microcontroller 560 by comparing them to a predefined rate zone limit (i.e., bradycardia, normal, atrial tachycardia, atrial fibrillation, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks).

Cardiac signals are also applied to the inputs of an analog-to-digital (ND) data acquisition system 590. The data acquisition system 590 is configured to acquire intracardiac electrogram signals, convert the raw analog data into a digital signal, and store the digital signals for later processing and/or telemetric transmission to an external device 602. The data acquisition system 590 is coupled to the right atrial lead 520, the LV lead 524, and the right ventricular lead 530 through the switch 574 to sample cardiac signals across any pair of desired electrodes. The microcontroller 560 is further coupled to a memory 594 by a suitable data/address bus 596, wherein the programmable operating parameters used by the microcontroller 560 are stored and modified, as required, in order to customize the operation of CRMD 10 to suit the needs of a particular patient. Such operating parameters define, for example, the amplitude or magnitude, pulse duration, electrode polarity, for both pacing pulses and impedance detection pulses as well as pacing rate, sensitivity, arrhythmia detection criteria, and the amplitude, waveshape and vector of each shocking pulse to be delivered to the patient's heart within each respective tier of therapy. Other pacing parameters include base rate, rest rate and circadian base rate.

Advantageously, the operating parameters of the implantable CRMD 10 may be non-invasively programmed into the memory 594 through a telemetry circuit 600 in telemetric communication with the external device 16, such as a programmer, transtelephonic transceiver or a diagnostic system analyzer. The telemetry circuit 600 is activated by the microcontroller by a control signal 606. The telemetry circuit 600 advantageously allows intracardiac electrograms and status information relating to the operation of CRMD 10 (as contained in the microcontroller 560 or memory 594) to be sent to the external device 16 through an established communication link 604. CRMD 10 further includes an accelerometer or other physiologic sensor 608, commonly referred to as a “rate-responsive” sensor because it is typically used to adjust pacing stimulation rate according to the exercise state of the patient. However, the physiological sensor 608 may further be used to detect changes in cardiac output, changes in the physiological condition of the heart, or diurnal changes in activity (e.g., detecting sleep and wake states) and to detect arousal from sleep. Accordingly, the microcontroller 560 responds by adjusting the various pacing parameters (such as rate, AV delay, VV delay, etc.) at which the atrial and ventricular pulse generators, 570 and 572, generate stimulation pulses. While shown as being included within CRMD 10, it is to be understood that the physiologic sensor 608 may also be external to CRMD 10, yet still be implanted within or carried by the patient. A common type of rate responsive sensor is an activity sensor incorporating an accelerometer or a piezoelectric crystal, which is mounted within the housing 540 of CRMD 10. Other types of physiologic sensors are also known, for example, sensors that sense the oxygen content of blood, respiration rate and/or minute ventilation, pH of blood, ventricular gradient, etc. The accelerometer may be positioned and configured to detect signals representative of diaphragmatic stimulation, i.e. to operate as a plethysmograph.

The CRMD additionally includes a battery 610, which provides operating power to all of the circuits shown in FIG. 12. The particular battery 610 used may depend on the capabilities of CRMD 10. If the system only provides low voltage therapy, a lithium iodine or lithium copper fluoride cell typically may be utilized. For CRMD 10, which employs shocking therapy, the battery 610 should be capable of operating at low current drains for long periods, and then be capable of providing high-current pulses (for capacitor charging) when the patient requires a shock pulse. The battery 610 should also have a predictable discharge characteristic so that elective replacement time can be detected. Accordingly, appropriate batteries are employed. As shown, the battery is connected to switch 574 via a set of selectable voltage multipliers 611 (including voltage dividers) such as the aforementioned voltage quarterers, halvers, doublers, triplers, etc.

As further shown in FIG. 12, CRMD 10 has an impedance measuring circuit 612, which is enabled by the microcontroller 560 via a control signal 614. Uses for an impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring respiration; and detecting the opening of heart valves, and detecting cardiogenic impedance, etc. The impedance measuring circuit 612 is advantageously coupled to the switch 674 so that any desired electrode may be used. Impedance may also be used for plethysmography.

In the case where CRMD 10 is intended to operate as an ICD device, it detects the occurrence of an arrhythmia, and automatically applies an appropriate electrical shock therapy to the heart aimed at terminating the detected arrhythmia. To this end, the microcontroller 560 further controls a shocking circuit 616 by way of a control signal 618. The shocking circuit 616 generates shocking pulses of low (up to 0.5 joules), moderate (0.5-10 joules) or high energy (11 to 40 joules or more), as controlled by the microcontroller 560. Such shocking pulses are applied to the heart of the patient through at least two shocking electrodes, and as shown in this embodiment, selected from the left atrial coil electrode 528, the RV coil electrode 536, and/or the SVC coil electrode 538. The housing 540 may act as an active electrode in combination with the RV electrode 536, or as part of a split electrical vector using the SVC coil electrode 538 or the left atrial coil electrode 528 (i.e., using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 7-40 joules or more), delivered asynchronously (since R-waves may be too disorganized), and pertaining exclusively to the treatment of fibrillation. Accordingly, the microcontroller 560 is capable of controlling synchronous or asynchronous delivery of shocking pulses.

An internal warning device 599 may be provided for generating perceptible warning signals to the patient via vibration, voltage or other methods.

Insofar as energy optimization is concerned, the microcontroller includes (in this example) an on-board voltage multiplier-based optimizer 601, which is operative to control or perform the various optimization procedures described above, alone or in combination with external programmer 16 (or other external system.) Depending upon the configuration, the set of multiplier/dividers 611 may be directly connected to the microcontroller 560 via one or more control lines. In this particular example, the on-board optimizer 601 includes a voltage multiplier-based pulse width determination system 603 operative to determine candidate pulse widths for selected voltage multipliers, each candidate pulse width corresponding to a lowest energy pulse sufficient to achieve capture within the tissues of the patient using the selected vector and using a voltage supplied by a corresponding voltage multiplier. The optimizer also includes a lowest operable voltage multiplier determination system 605 operative to determine a lowest operable voltage multiplier based on the candidate minimum energy pulse widths determined by system 603. A voltage multiplier-based stimulation control system 607 is also provided that is operative to control generation of pulses using the lowest operable voltage multiplier for delivery along the selected vector.

The exemplary microcontroller also provides: a phrenic stimulation detector/controller 609 for controlling the phrenic stimulation functions discussed above; a bipolar anodal/cathodal stimulation controller 613 for controlling the concurrent anodal/cathodal stimulation functions discussed above; an automatic safety margin adjustment controller 615 for controlling the safety margin adjustment functions discussed above such as lowering the margin following the acute post-implant phase; and an interventricular pacing delay optimization controller 617 for controlling interventricular pacing delay optimization functions. A biventricular/CRT controller 619 controls biventricular pacing therapy, CRT or other forms of therapy. A warning/diagnostics controller 621 controls the storage of diagnostics data for transmission to the external programmer 16 and the generation of any warning signals, if warranted. Diagnostic data can be stored within memory 594. Warning signals may be generated via warning device 599.

Depending upon the implementation, the various components of the microcontroller may be implemented as separate software modules or the modules may be combined to permit a single module to perform multiple functions. In addition, although shown as being components of the microcontroller, some or all of these components may be implemented separately from the microcontroller, using application specific integrated circuits (ASICs) or the like.

As noted, at least some of the techniques described herein can be performed by (or under the control of) an external device. For the sake of completeness, a detailed description of an exemplary device programmer will now be provided.

### Exemplary Device Programmer

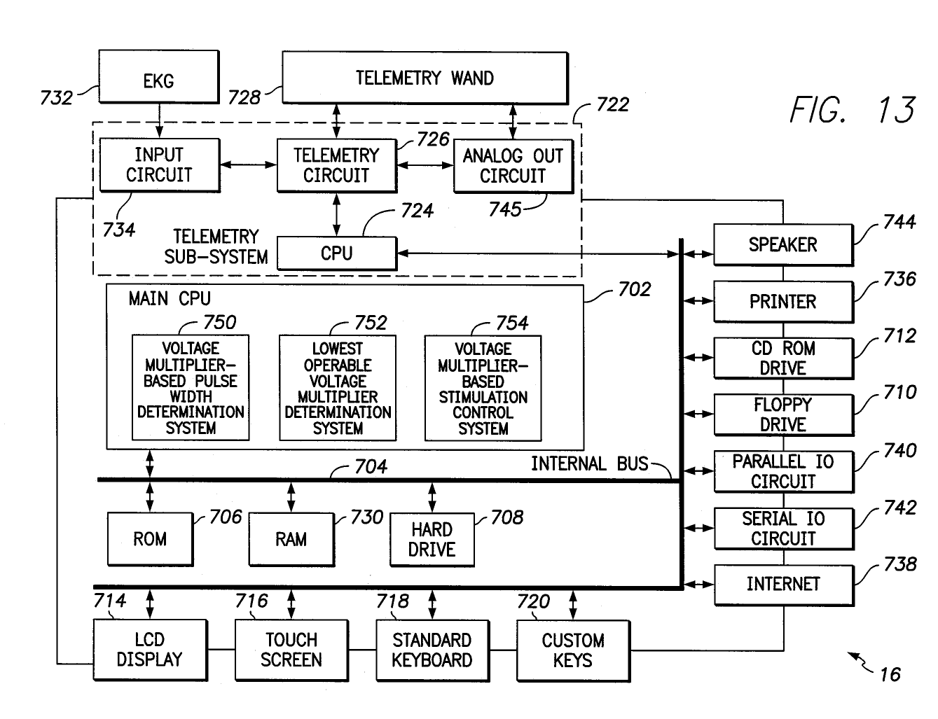

FIG. 13 illustrates pertinent components of an external programmer 16 for use in programming the CRMD of FIGS. 11 and 12 and for performing the above-described vector selection and optimization techniques. For the sake of completeness, other device programming functions are also described herein. Generally, the programmer permits a physician, clinician or other user to program the operation of the implanted device and to retrieve and display information received from the implanted device such as IEGM data and device diagnostic data. Additionally, the external programmer can be optionally equipped to receive and display electrocardiogram (EKG) data from separate external EKG leads that may be attached to the patient. Depending upon the specific programming of the external programmer, programmer 16 may also be capable of processing and analyzing data received from the implanted device and from the EKG leads to, for example, render preliminary diagnosis as to medical conditions of the patient or to the operations of the implanted device.

Now considering the components of programmer 16, operations of the programmer are controlled by a CPU 702, which may be a generally programmable microprocessor or microcontroller or may be a dedicated processing device such as an application specific integrated circuit (ASIC) or the like. Software instructions to be performed by the CPU are accessed via an internal bus 704 from a read only memory (ROM) 706 and random access memory 730. Additional software may be accessed from a hard drive 708, floppy drive 710, and CD ROM drive 712, or other suitable permanent mass storage device. Depending upon the specific implementation, a basic input output system (BIOS) is retrieved from the ROM by CPU at power up. Based upon instructions provided in the BIOS, the CPU “boots up” the overall system in accordance with well-established computer processing techniques.

Once operating, the CPU displays a menu of programming options to the user via an LCD display 714 or other suitable computer display device. To this end, the CPU may, for example, display a menu of specific programmable parameters of the implanted device to be programmed or may display a menu of types of diagnostic data to be retrieved and displayed. In response thereto, the physician enters various commands via either a touch screen 716 overlaid on the LCD display or through a standard keyboard 718 supplemented by additional custom keys 720, such as an emergency VVI (EVVI) key. The EVVI key sets the implanted device to a safe WI mode with high pacing outputs. This ensures life sustaining pacing operation in nearly all situations but by no means is it desirable to leave the implantable device in the EVVI mode at all times.

Once all pacing leads are mounted and the pacing device is implanted, the various parameters are programmed. Typically, the physician initially controls the programmer 16 to retrieve data stored within any implanted devices and to also retrieve EKG data from EKG leads, if any, coupled to the patient. To this end, CPU 702 transmits appropriate signals to a telemetry subsystem 722, which provides components for directly interfacing with the implanted devices, and the EKG leads. Telemetry subsystem 722 includes its own separate CPU 724 for coordinating the operations of the telemetry subsystem. Main CPU 702 of programmer communicates with telemetry subsystem CPU 724 via internal bus 704. Telemetry subsystem additionally includes a telemetry circuit 726 connected to telemetry wand 728, which, in turn, receives and transmits signals electromagnetically from a telemetry unit of the implanted device. The telemetry wand is placed over the chest of the patient near the implanted device to permit reliable transmission of data between the telemetry wand and the implanted device. Herein, the telemetry subsystem is shown as also including an input circuit 734 for receiving surface EKG signals from a surface EKG system 732. In other implementations, the EKG circuit is not regarded as a portion of the telemetry subsystem but is regarded as a separate component.

Typically, at the beginning of the programming session, the external programming device controls the implanted devices via appropriate signals generated by the telemetry wand to output all previously recorded patient and device diagnostic information. Patient diagnostic information includes, for example, recorded IEGM data and statistical patient data such as the percentage of paced versus sensed heartbeats. Device diagnostic data includes, for example, information representative of the operation of the implanted device such as lead impedances, battery voltages, battery recommended replacement time (RRT) information and the like. Data retrieved from the CRMD also includes the data stored within the recalibration database of the CRMD (assuming the CRMD is equipped to store that data.) Data retrieved from the implanted devices is stored by external programmer 16 either within a random access memory (RAM) 730, hard drive 708 or within a floppy diskette placed within floppy drive 710. Additionally, or in the alternative, data may be permanently or semi-permanently stored within a compact disk (CD) or other digital media disk, if the overall system is configured with a drive for recording data onto digital media disks, such as a write once read many (WORM) drive.

Once all patient and device diagnostic data previously stored within the implanted devices is transferred to programmer 16, the implanted devices may be further controlled to transmit additional data in real time as it is detected by the implanted devices, such as additional IEGM data, lead impedance data, and the like. Additionally, or in the alternative, telemetry subsystem 722 receives EKG signals from EKG leads 732 via an EKG processing circuit 734. As with data retrieved from the implanted device itself, signals received from the EKG leads are stored within one or more of the storage devices of the external programmer. Typically, EKG leads output analog electrical signals representative of the EKG. Accordingly, EKG circuit 734 includes analog to digital conversion circuitry for converting the signals to digital data appropriate for further processing within the programmer. Depending upon the implementation, the EKG circuit may be configured to convert the analog signals into event record data for ease of processing along with the event record data retrieved from the implanted device. Typically, signals received from the EKG leads are received and processed in real time.

Thus, the programmer receives data both from the implanted devices and from optional external EKG leads. Data retrieved from the implanted devices includes parameters representative of the current programming state of the implanted devices. Under the control of the physician, the external programmer displays the current programmable parameters and permits the physician to reprogram the parameters. To this end, the physician enters appropriate commands via any of the aforementioned input devices and, under control of CPU 702, the programming commands are converted to specific programmable parameters for transmission to the implanted devices via telemetry wand 728 to thereby reprogram the implanted devices. Prior to reprogramming specific parameters, the physician may control the external programmer to display any or all of the data retrieved from the implanted devices or from the EKG leads, including displays of EKGs, IEGMs, and statistical patient information. Any or all of the information displayed by programmer may also be printed using a printer 736.

Programmer/monitor 16 also includes an internet connection 738 to permit direct transmission of data to other programmers via the public switched telephone network (PSTN) or other interconnection line, such as a T1 line, fiber optic cable, Wi-Fi, cellular network, etc. Depending upon the implementation, the modem may be connected directly to internal bus 704 may be connected to the internal bus via either a parallel port 740 or a serial port 742. Other peripheral devices may be connected to the external programmer via parallel port 740 or a serial port 742 as well. Although one of each is shown, a plurality of input output (IO) ports might be provided. A speaker 744 is included for providing audible tones to the user, such as a warning beep in the event improper input is provided by the physician. Telemetry subsystem 722 additionally includes an analog output circuit 745 for controlling the transmission of analog output signals, such as IEGM signals output to an EKG machine or chart recorder.

Insofar as vector selection and energy optimization is concerned, main CPU 702 may include components corresponding to any or all of the components of the on-board optimizer of the CRMD (FIG. 12) but configured to operate based on data received from the CRMD. Three exemplary components are specifically shown in FIG. 13 including a voltage multiplier-based pulse width determination system 750 operative to determine candidate pulse widths for selected voltage multipliers of the CRMD, each candidate pulse width corresponding to a lowest energy pulse sufficient to achieve capture within the tissues of the patient using the selected vector and using a voltage supplied by a corresponding voltage multiplier of the CRMD. Main CPU also includes a lowest operable voltage multiplier determination system 752 operative to determine a lowest operable voltage multiplier of the CRMD based on the candidate minimum energy pulse widths. A voltage multiplier-based stimulation control system 754 is also provided, which is operative to control the CRMD to generate stimulation pulses using the lowest operable voltage multiplier for delivery along the selected vector(s). Depending upon the implementation, the various components of the CPU may be implemented as separate software modules or the modules may be combined to permit a single module to perform multiple functions. In addition, although shown as being components of the CPU, some or all of these components may be implemented separately, using ASICs or the like.

The descriptions provided herein with respect to FIG. 13 are intended merely to provide an overview of the operation of programmer and are not intended to describe in detail every feature of the hardware and software of the programmer and is not intended to provide an exhaustive list of the functions performed by the programmer.

In general, while the invention has been described with reference to particular embodiments, modifications can be made thereto without departing from the scope of the invention. Note also that the term “including” as used herein is intended to be inclusive, i.e. “including but not limited to.”

What is claimed is:

1. A method for use with an implantable medical device equipped for generating electrical stimulation pulses for delivery along stimulation vectors through tissues of a patient in which the device is implanted, the device provided with a set of voltage multipliers connected to a voltage source for providing voltages for the stimulation pulses, the method comprising:
determining candidate pulse widths for selected voltage multipliers and for selected stimulation vectors, each candidate pulse width corresponding to a lowest energy pulse sufficient to achieve capture within the tissues of the patient using a selected vector and using a voltage supplied by a corresponding voltage multiplier;
determining a lowest operable voltage multiplier for the selected vector based on the candidate minimum energy pulse widths; and
controlling generation of stimulation pulses using the lowest operable voltage multiplier for delivery along the selected vector.

2. The method of claim 1 wherein determining candidate pulse widths for selected voltage multipliers comprises:
determining rheobase (Rho) and chronaxie (Chron) values of tissues along the selected stimulation vector; and
for a selected multiplier value (Mutt) corresponding to a particular voltage multiplier, determining a candidate pulse width (PWopt) for the selected vector based at least on the rheobase (Rho) and chronaxie (Chron) values, the multiplier value (Mutt) and a base voltage (Vbat) of the voltage source of the device.

3. The method of claim 2 wherein determining the rheobase (Rho) and chronaxie (Chron) values for the selected vector comprises:
delivering stimulation pulses using at least two different pulse widths (PW1, PW2) along the selected vector, wherein the pulse widths are selected from within a range of programmable pulse widths;
measuring corresponding capture voltage thresholds (Vth1, Vth2); and
determining rheobase (Rho) and chronaxie (Chron) from the two different pulse widths (PW1, PW2) and the corresponding capture voltage thresholds (Vth1, Vth2).

4. The method of claim 3 wherein determining rheobase (Rho) comprises determining:
$$Rho = \frac{{V}_{th 2} - \frac{{PW}_{1}}{{PW}_{2}} \cdot {V}_{th 1}}{1 - \frac{{PW}_{1}}{{PW}_{2}}} .$$

5. The method of claim 4 wherein determining chronaxie (Chron) comprises determining:
$$Chron = \frac{( {V}_{th 1} - Rho ) \cdot {PW}_{1}}{Rho} .$$

6. The method of claim 2 wherein determining a candidate pulse width (PWopt) for the selected vector and for a selected multiplier value (Mutt) comprises determining:
$${PW}_{opt} = \frac{Chron}{\frac{{V}_{stim}}{Margin \cdot Rho} - 1} {V}_{stim}$$
wherein Margin is a safety margin factor and Vstim is Mult times Vbat.

7. The method of claim 2 wherein the selectable voltage multiplier values of the device include one or more of ⅓, ¼, ½, 1, 2, 3 and 4.

8. The method of claim 2 wherein determining the lowest operable voltage multiplier for a selected vector comprises:
beginning with a lowest selected multiplier value (Mutt), determining whether the candidate pulse width (PWopt) for that voltage multiplier is within a programmable range of pulse widths;
if a currently selected multiplier value (Mutt) has a candidate pulse width (PWopt) within the programmable range of pulse widths, identifying the corresponding voltage multiplier as the lowest operable voltage multiplier; and
if the currently selected multiplier value (Mutt) does not have a candidate pulse width (PWopt) within the programmable range of pulse widths, repeating the procedure with a next higher multiplier value.

9. The method of claim 8 wherein the range of programmable pulse widths comprise a range from 0.05 milliseconds (ms) to 1.0 ms, inclusive.

10. The method of claim 8 wherein if none of the voltage multipliers achieves a candidate pulse width (PWopt) within the range of programmable pulse widths for the selected vector, generating an indicator warning that the selected vector is not suitable.

11. The method of claim 8 wherein if none of the voltage multipliers achieves a candidate pulse width (PWopt) within the range of programmable pulse widths for any programmable vector of the device, generating an indicator warning that none of the programmable vectors of the device is suitable.

12. The method of claim 2 further including determining a pulse charge corresponding to the candidate pulse width (PWopt) for a selected voltage multiplier.

13. The method of claim 12 wherein determining the pulse charge corresponding to the candidate pulse width (PW) comprises:
determining the impedance (R) of the selected vector; and
determining the pulse charge corresponding to the candidate pulse width (PWopt) for the vector using the lowest operable voltage multiplier (Mutt) for that vector and the determined impedance (R).

14. The method of claim 13 wherein the pulse charge is determined using:
$${Q}_{bat\_opt} = Mult \cdot \frac{{V}_{stim} \cdot {PW}_{opt}}{R}$$
wherein Vstim is Mult times the voltage (Vbat) of the voltage source.

15. The method of claim 12 further including converting the pulse charge to one or more of: a relative vector combination efficiency; a charge drawn from battery per pulse value; an energy from battery per pulse value; a microampere value of battery current drain associated with pacing; an incremental increase in current drain value; and an incremental expected decrease in longevity value.

16. The method of claim 12 wherein a lowest operable voltage multiplier and corresponding pulse charge are determined for each of a plurality of stimulation vectors.

17. The method of claim 16 further including selecting a particular vector from among the plurality of stimulation vectors based on the pulse charge values.

18. The method of claim 17 wherein the vector having the lowest pulse charge value is selected and wherein generation of stimulation pulses is controlled to use the candidate pulse width corresponding to the lowest operable voltage multiplier for that vector.

19. The method of claim 17 wherein selecting a particular vector from among the plurality of stimulation vectors is performed to select a vector from among a set of vectors capable of both anodal and cathodal stimulation.

20. The method of claim 1 further including determining whether delivery of stimulation pulses using the candidate pulse width corresponding to the lowest operable voltage multiplier along the selected vector triggers phrenic nerve stimulation within the patient and, if so, rejecting that vector.

21. The method of claim 1 wherein all of the steps are performed by the implantable device.

22. The method of claim 1 wherein at least one of the steps is performed by an external system equipped to communicate with the implantable device.

23. The method of claim 22 wherein the external system is a programmer device.

24. The method of claim 24 wherein the programmer device is equipped with a “one button” energy optimization function that controls the operation of each of said steps for each of a set of stimulation vectors.

25. The method of claim 1 further including resetting a safety margin associated with stimulation pulses based on energy optimization for setting one or more of a Vstim value and a PWopt value for the selected stimulation vector where Vstim is a stimulation voltage value and PWopt is a pulse width value.

26. The method of claim 25 wherein the safety margin is set to a first value during an initial post-implant acute phase and to a second value during a subsequent chronic implant phase.

27. The method of claim 1 further including assessing interventricular conduction delays and wherein controlling generation of stimulation pulses is also based, at least in part, on the interventricular conduction delays.

28. The method of claim 1 wherein a selected stimulation vector can include a combination of vectors.

29. A system within an implantable medical device equipped for generating electrical stimulation pulses for delivery along stimulation vectors through tissues of a patient in which the device is implanted, the device provided with a set of voltage multipliers connected to a voltage source for providing voltages for the stimulation pulses, the system comprising:
a voltage multiplier-based pulse width determination system operative for a selected stimulation vector to determine candidate pulse widths for selected voltage multipliers, each candidate pulse width corresponding to a lowest energy pulse sufficient to achieve capture within the tissues of the patient using the selected vector and using a voltage supplied by a corresponding voltage multiplier;
a lowest operable voltage multiplier determination system operative to determine a lowest operable voltage multiplier based on the candidate minimum energy pulse widths; and
a voltage multiplier-based stimulation control system operative to control generation of stimulation pulses using the lowest operable voltage multiplier for delivery along the selected vector.

30. An external system for use with an implantable medical device equipped for generating electrical stimulation pulses for delivery along stimulation vectors through tissues of a patient in which the device is implanted, the implantable device provided with a set of voltage multipliers connected to a voltage source for providing voltages for the stimulation pulses, the external system comprising:
a voltage multiplier-based pulse width determination system operative for a selected stimulation vector to determine candidate pulse widths for selected voltage multipliers, each candidate pulse width corresponding to a lowest energy pulse sufficient to achieve capture within the tissues of the patient using the selected vector and using a voltage supplied by a corresponding voltage multiplier;
a lowest operable voltage multiplier determination system operative to determine a lowest operable voltage multiplier based on the candidate minimum energy pulse widths; and
a voltage multiplier-based stimulation control system operative to generate control signals for sending to the implantable device for controlling the device to generate stimulation pulses using the lowest operable voltage multiplier for delivery along the selected vector.

31. A system for use with an implantable medical device equipped for generating electrical stimulation pulses for delivery along stimulation vectors through tissues of a patient in which the device is implanted, the device provided with a set of voltage multipliers connected to a voltage source for providing voltages for the stimulation pulses, the system comprising:
means for determining candidate pulse widths for selected voltage multipliers and for a selected stimulation vector, each candidate pulse width corresponding to a lowest energy pulse sufficient to achieve capture within the tissues of the patient using the selected vector and using a voltage supplied by a corresponding voltage multiplier;
means for determining a lowest operable voltage multiplier for the selected vector based on the candidate minimum energy pulse widths; and
means for controlling generation of stimulation pulses using the lowest operable voltage multiplier for delivery along the selected vector.
